SEQUENCE LISTING (64 sequences)

SEQ ID NO: 1 - DNA, 109 bp, synthetic construct
  source 1..109: mol_type = other DNA
ttaattaagctgccttctgcggggcttgccttctggccatgcccttcttctctcccttgcacctgtacctcttgnnnnnnnngtctttgaataaagcctgagtaggaag

SEQ ID NO: 2 - DNA, 121 bp, synthetic construct
  source 1..121: mol_type = other DNA
ttaattaagctgccttctgcggggcttgccttctggccatgcccttcttctctcccttgcacctgtacctcttgnnnnnnnnnnnnnnnnnnnngtctttgaataaagcctgagtaggaag

SEQ ID NO: 3 - skipped

SEQ ID NO: 4 - DNA, 171 bp, synthetic construct
  source 1..171: mol_type = other DNA
ttttttttttttttttttttttttttttttttttttttttttttttttttttttttttttttttttttttttttttttttttttttttttttttttttttttttttttttttttttttttcttcctactcaggctttattcaaagacgcgatgttcaagaggtacaggtgc

SEQ ID NO: 5 - skipped

SEQ ID NO: 6 - DNA, 171 bp, synthetic construct
  source 1..171: mol_type = other DNA
ttttttttttttttttttttttttttttttttttttttttttttttttttttttttttttttttttttttttttttttttttttttttttttttttttttttttttttttttttttttttcttcctactcaggctttattcaaagacggtgttgacaagaggtacaggtgc

SEQ ID NO: 7 - skipped

SEQ ID NO: 8 - DNA, 171 bp, synthetic construct
  source 1..171: mol_type = other DNA
ttttttttttttttttttttttttttttttttttttttttttttttttttttttttttttttttttttttttttttttttttttttttttttttttttttttttttttttttttttttttcttcctactcaggctttattcaaagacggatcgaacaagaggtacaggtgc

SEQ ID NO: 9 - skipped

SEQ ID NO: 10 - DNA, 171 bp, synthetic construct
  source 1..171: mol_type = other DNA
ttttttttttttttttttttttttttttttttttttttttttttttttttttttttttttttttttttttttttttttttttttttttttttttttttttttttttttttttttttttttcttcctactcaggctttattcaaagacggatgagacaagaggtacaggtgc

SEQ ID NO: 11 - skipped

SEQ ID NO: 12 - DNA, 171 bp, synthetic construct
  source 1..171: mol_type = other DNA
ttttttttttttttttttttttttttttttttttttttttttttttttttttttttttttttttttttttttttttttttttttttttttttttttttttttttttttttttttttttttcttcctactcaggctttattcaaagacgaggtgatcaagaggtacaggtgc

SEQ ID NO: 13 - skipped

SEQ ID NO: 14 - DNA, 171 bp, synthetic construct
  source 1..171: mol_type = other DNA
ttttttttttttttttttttttttttttttttttttttttttttttttttttttttttttttttttttttttttttttttttttttttttttttttttttttttttttttttttttttttcttcctactcaggctttattcaaagacagcggattcaagaggtacaggtgc

SEQ ID NO: 15 - skipped

SEQ ID NO: 16 - DNA, 171 bp, synthetic construct
  source 1..171: mol_type = other DNA
ttttttttttttttttttttttttttttttttttttttttttttttttttttttttttttttttttttttttttttttttttttttttttttttttttttttttttttttttttttttttcttcctactcaggctttattcaaagacagcgacatcaagaggtacaggtgc

SEQ ID NO: 17 - skipped

SEQ ID NO: 18 - DNA, 171 bp, synthetic construct
  source 1..171: mol_type = other DNA
ttttttttttttttttttttttttttttttttttttttttttttttttttttttttttttttttttttttttttttttttttttttttttttttttttttttttttttttttttttttttcttcctactcaggctttattcaaagacagcgaagacaagaggtacaggtgc

SEQ ID NO: 19 - DNA, 20 bp, synthetic construct
  source 1..20: mol_type = other DNA
tcgtagatgtccgcagtctc

SEQ ID NO: 20 - DNA, 183 bp, synthetic construct
  source 1..183: mol_type = other DNA
ttttttttttttttttttttttttttttttttttttttttttttttttttttttttttttttttttttttttttttttttttttttttttttttttttttttttttttttttttttttttcttcctactcaggctttattcaaagacgagactgcggacatctacgacaagaggtacaggtgc

SEQ ID NO: 21 - DNA, 20 bp, synthetic construct
  source 1..20: mol_type = other DNA
tctcgcttacctcactcgaa

SEQ ID NO: 22 - DNA, 183 bp, synthetic construct
  source 1..183: mol_type = other DNA
ttttttttttttttttttttttttttttttttttttttttttttttttttttttttttttttttttttttttttttttttttttttttttttttttttttttttttttttttttttttttcttcctactcaggctttattcaaagacttcgagtgaggtaagcgagacaagaggtacaggtgc

SEQ ID NO: 23 - DNA, 20 bp, synthetic construct
  source 1..20: mol_type = other DNA
ttcgtagatgtcggtccgga

SEQ ID NO: 24 - DNA, 183 bp, synthetic construct
  source 1..183: mol_type = other DNA
ttttttttttttttttttttttttttttttttttttttttttttttttttttttttttttttttttttttttttttttttttttttttttttttttttttttttttttttttttttttttcttcctactcaggctttattcaaagactccggaccgacatctacgaacaagaggtacaggtgc

SEQ ID NO: 25 - DNA, 20 bp, synthetic construct
  source 1..20: mol_type = other DNA
ttcgaatcctatcacgctcc

SEQ ID NO: 26 - DNA, 183 bp, synthetic construct
  source 1..183: mol_type = other DNA
ttttttttttttttttttttttttttttttttttttttttttttttttttttttttttttttttttttttttttttttttttttttttttttttttttttttttttttttttttttttttcttcctactcaggctttattcaaagacggagcgtgataggattcgaacaagaggtacaggtgc

SEQ ID NO: 27 - DNA, 20 bp, synthetic construct
  source 1..20: mol_type = other DNA
atccttcctaaccgagcatt

SEQ ID NO: 28 - DNA, 183 bp, synthetic construct
  source 1..183: mol_type = other DNA
ttttttttttttttttttttttttttttttttttttttttttttttttttttttttttttttttttttttttttttttttttttttttttttttttttttttttttttttttttttttttcttcctactcaggctttattcaaagacaatgctcggttaggaaggatcaagaggtacaggtgc

SEQ ID NO: 29 - DNA, 20 bp, synthetic construct
  source 1..20: mol_type = other DNA
cgcgtcctatcttatgttcc

SEQ ID NO: 30 - DNA, 183 bp, synthetic construct
  source 1..183: mol_type = other DNA
ttttttttttttttttttttttttttttttttttttttttttttttttttttttttttttttttttttttttttttttttttttttttttttttttttttttttttttttttttttttttcttcctactcaggctttattcaaagacggaacataagataggacgcgcaagaggtacaggtgc

SEQ ID NO: 31 - DNA, 20 bp, synthetic construct
  source 1..20: mol_type = other DNA
atatccgcagtcaacgttag

SEQ ID NO: 32 - DNA, 183 bp, synthetic construct
  source 1..183: mol_type = other DNA
ttttttttttttttttttttttttttttttttttttttttttttttttttttttttttttttttttttttttttttttttttttttttttttttttttttttttttttttttttttttttcttcctactcaggctttattcaaagacctaacgttgactgcggatatcaagaggtacaggtgc

SEQ ID NO: 33 - DNA, 20 bp, synthetic construct
  source 1..20: mol_type = other DNA
gctgagtcggttgtctgtaa

SEQ ID NO: 34 - DNA, 183 bp, synthetic construct
  source 1..183: mol_type = other DNA
ttttttttttttttttttttttttttttttttttttttttttttttttttttttttttttttttttttttttttttttttttttttttttttttttttttttttttttttttttttttttcttcctactcaggctttattcaaagacttacagacaaccgactcagccaagaggtacaggtgc

SEQ ID NO: 35 - DNA, 20 bp, synthetic construct
  source 1..20: mol_type = other DNA
ccattctattatgctaccgt

SEQ ID NO: 36 - DNA, 183 bp, synthetic construct
  source 1..183: mol_type = other DNA
ttttttttttttttttttttttttttttttttttttttttttttttttttttttttttttttttttttttttttttttttttttttttttttttttttttttttttttttttttttttttcttcctactcaggctttattcaaagacacggtagcataatagaatggcaagaggtacaggtgc

SEQ ID NO: 37 - DNA, 20 bp, synthetic construct
  source 1..20: mol_type = other DNA
aacacggccagttactaaca

SEQ ID NO: 38 - DNA, 183 bp, synthetic construct
  source 1..183: mol_type = other DNA
ttttttttttttttttttttttttttttttttttttttttttttttttttttttttttttttttttttttttttttttttttttttttttttttttttttttttttttttttttttttttcttcctactcaggctttattcaaagactgttagtaactggccgtgttcaagaggtacaggtgc

SEQ ID NO: 39 - DNA, 20 bp, synthetic construct
  source 1..20: mol_type = other DNA
gcgtctgaagaacctgtata

SEQ ID NO: 40 - DNA, 183 bp, synthetic construct
  source 1..183: mol_type = other DNA
ttttttttttttttttttttttttttttttttttttttttttttttttttttttttttttttttttttttttttttttttttttttttttttttttttttttttttttttttttttttttcttcctactcaggctttattcaaagactatacaggttcttcagacgccaagaggtacaggtgc

SEQ ID NO: 41 - DNA, 20 bp, synthetic construct
  source 1..20: mol_type = other DNA
tatgccatcctcgtcctatt

SEQ ID NO: 42 - DNA, 183 bp, synthetic construct
  source 1..183: mol_type = other DNA
ttttttttttttttttttttttttttttttttttttttttttttttttttttttttttttttttttttttttttttttttttttttttttttttttttttttttttttttttttttttttcttcctactcaggctttattcaaagacaataggacgaggatggcatacaagaggtacaggtgc

SEQ ID NO: 43 - DNA, 20 bp, synthetic construct
  source 1..20: mol_type = other DNA
tcaatctcactggctggaac

SEQ ID NO: 44 - DNA, 183 bp, synthetic construct
  source 1..183: mol_type = other DNA
ttttttttttttttttttttttttttttttttttttttttttttttttttttttttttttttttttttttttttttttttttttttttttttttttttttttttttttttttttttttttcttcctactcaggctttattcaaagacgttccagccagtgagattgacaagaggtacaggtgc

SEQ ID NO: 45 - DNA, 20 bp, synthetic construct
  source 1..20: mol_type = other DNA
ttccagtccgctacgtaaca

SEQ ID NO: 46 - DNA, 183 bp, synthetic construct
  source 1..183: mol_type = other DNA
ttttttttttttttttttttttttttttttttttttttttttttttttttttttttttttttttttttttttttttttttttttttttttttttttttttttttttttttttttttttttcttcctactcaggctttattcaaagactgttacgtagcggactggaacaagaggtacaggtgc

SEQ ID NO: 47 - DNA, 20 bp, synthetic construct
  source 1..20: mol_type = other DNA
ccttagtctcaccgagccga

SEQ ID NO: 48 - DNA, 183 bp, synthetic construct
  source 1..183: mol_type = other DNA
ttttttttttttttttttttttttttttttttttttttttttttttttttttttttttttttttttttttttttttttttttttttttttttttttttttttttttttttttttttttttcttcctactcaggctttattcaaagactcggctcggtgagactaaggcaagaggtacaggtgc

SEQ ID NO: 49 - DNA, 20 bp, synthetic construct
  source 1..20: mol_type = other DNA
gcgttcaatgttcacctgtc

SEQ ID NO: 50 - DNA, 183 bp, synthetic construct
  source 1..183: mol_type = other DNA
ttttttttttttttttttttttttttttttttttttttttttttttttttttttttttttttttttttttttttttttttttttttttttttttttttttttttttttttttttttttttcttcctactcaggctttattcaaagacgacaggtgaacattgaacgccaagaggtacaggtgc

SEQ ID NO: 51 - DNA, 101 bp, synthetic construct
  source 1..101: mol_type = other DNA
ttaattaagctgccttctgcggggcttgccttctggccatgcccttcttctctcccttgcacctgtacctcttggtctttgaataaagcctgagtaggaag

SEQ ID NO: 52 - DNA, 74 bp, synthetic construct
  source 1..74: mol_type = other DNA
ttaattaagctgccttctgcggggcttgccttctggccatgcccttcttctctcccttgcacctgtacctcttg

SEQ ID NO: 53 - DNA, 27 bp, synthetic construct
  source 1..27: mol_type = other DNA
gtctttgaataaagcctgagtaggaag

SEQ ID NO: 54 - DNA, 171 bp, synthetic construct
  source 1..171: mol_type = other DNA
ttttttttttttttttttttttttttttttttttttttttttttttttttttttttttttttttttttttttttttttttttttttttttttttttttttttttttttttttttttttttcttcctactcaggctttattcaaagacttcgcgatcaagaggtacaggtgc

SEQ ID NO: 55 - DNA, 171 bp, synthetic construct
  source 1..171: mol_type = other DNA
ttttttttttttttttttttttttttttttttttttttttttttttttttttttttttttttttttttttttttttttttttttttttttttttttttttttttttttttttttttttttcttcctactcaggctttattcaaagactcggtagacaagaggtacaggtgc

SEQ ID NO: 56 - DNA, 171 bp, synthetic construct
  source 1..171: mol_type = other DNA
ttttttttttttttttttttttttttttttttttttttttttttttttttttttttttttttttttttttttttttttttttttttttttttttttttttttttttttttttttttttttcttcctactcaggctttattcaaagactcgacgaacaagaggtacaggtgc

SEQ ID NO: 57 - DNA, 171 bp, synthetic construct
  source 1..171: mol_type = other DNA
ttttttttttttttttttttttttttttttttttttttttttttttttttttttttttttttttttttttttttttttttttttttttttttttttttttttttttttttttttttttttcttcctactcaggctttattcaaagactcgaacgacaagaggtacaggtgc

SEQ ID NO: 58 - DNA, 171 bp, synthetic construct
  source 1..171: mol_type = other DNA
ttttttttttttttttttttttttttttttttttttttttttttttttttttttttttttttttttttttttttttttttttttttttttttttttttttttttttttttttttttttttcttcctactcaggctttattcaaagaccggatagacaagaggtacaggtgc

SEQ ID NO: 59 - DNA, 171 bp, synthetic construct
  source 1..171: mol_type = other DNA
ttttttttttttttttttttttttttttttttttttttttttttttttttttttttttttttttttttttttttttttttttttttttttttttttttttttttttttttttttttttttcttcctactcaggctttattcaaagacgttcggttcaagaggtacaggtgc

SEQ ID NO: 60 - DNA, 171 bp, synthetic construct
  source 1..171: mol_type = other DNA
ttttttttttttttttttttttttttttttttttttttttttttttttttttttttttttttttttttttttttttttttttttttttttttttttttttttttttttttttttttttttcttcctactcaggctttattcaaagacgtcggtaacaagaggtacaggtgc

SEQ ID NO: 61 - DNA, 171 bp, synthetic construct
  source 1..171: mol_type = other DNA
ttttttttttttttttttttttttttttttttttttttttttttttttttttttttttttttttttttttttttttttttttttttttttttttttttttttttttttttttttttttttcttcctactcaggctttattcaaagacgccgatatcaagaggtacaggtgc

SEQ ID NO: 62 - DNA, 20 bp, synthetic construct
  source 1..20: mol_type = other DNA
aagcgcaaagtgtagttaat

SEQ ID NO: 63 - DNA, 21 bp, synthetic construct
  source 1..21: mol_type = other DNA
ttcctactcaggctttattca

SEQ ID NO: 64 - DNA, 20 bp, synthetic construct
  source 1..20: mol_type = other DNA
tctggccatgcccttcttct

(19) United States
(12) Patent Application Publication
(10) Pub. No.: US 2025/0092450 A1
(43) Pub. Date: Mar. 20, 2025

(54) IDENTIFICATION OF NANOPARTICLES FOR PREFERENTIAL TISSUE OR CELL TARGETING

(71) Applicant: Beam Therapeutics Inc., Cambridge, MA (US)

(72) Inventors: Nicholas JUSTICE, Cambridge, MA (US); Courtney WONG, Cambridge, MA (US); David BRYSON, Cambridge, MA (US); Valentina Laclare MCENEANY, Cambridge, MA (US); Maya SEN, Cambridge, MA (US); Cory D. SAGO, Cambridge, MA (US); Kathryn MURRAY, Cambridge, MA (US)

(21) Appl. No.: 18/960,564

(22) Filed: Nov. 26, 2024

Related U.S. Application Data
(63) Continuation of application No. PCT/US2023/067543, filed on May 26, 2023.
(60) Provisional application No. 63/346,808, filed on May 27, 2022.

(51) Int. Cl. C12Q 1/6851; C12N 9/22; C12N 15/10; C12N 15/11; C12N 15/88; C12Q 1/686

(52) U.S. Cl. CPC C12Q 1/6851; C12N 9/22; C12N 15/1096; C12N 15/111; C12N 15/88; C12Q 1/686; C12N 2310/20

(57) ABSTRACT

Provided herein includes high-throughput methods for in vivo screening nanoparticles (e.g., lipid nanoparticles) for preferential delivery to a target tissue or cell type. Also provided are compositions used in the high-throughput nanoparticle screening method.

### CROSS REFERENCE TO RELATED APPLICATIONS

The present application is a Continuation of International Application Number PCT/US2023/067543 filed on May 26, 2023, which claims priority to U.S. Provisional Application No. 63/346,808, filed on May 27, 2022; the contents of each of which are incorporated herein by reference in their entirety.

### CROSS REFERENCE TO SEQUENCE LISTING

The present application is being filed with an electronically filed Sequence Listing in XML format. The sequence listing file entitled BEM-016WO1_SL.XML, was created on May 18, 2023, and is 57,089 bytes in size; the information in electronic format of the Sequence Listing is incorporated herein by reference; in its entirely.

### BACKGROUND

Nanoparticles such as lipid nanoparticles (LNPs) have been used to deliver various nucleic acid based therapeutics. Successful nanoparticle based drug delivery requires specific tissue targeting as well as efficient intracellular transfection. Increasingly, research laboratories are fabricating libraries of novel nanoparticles, engineering both new biomaterial structures and composition ratios of multicomponent systems. Yet, there remains a need for efficient and reliable methods for screening and identifying nanoparticles (e.g., LNPs) that can specifically or preferentially target tissues or cells of clinical significance in vivo.

### SUMMARY OF THE INVENTION

The present invention provides, among other things, an improved method for screening nanoparticles (e.g., lipid nanoparticles) for preferential delivery to a target tissue or cell. The methods and compositions described herein provide several advantages over the prior art methods, as the present methods are robust and sensitive, and allow testing of multiple nanoparticles simultaneously, while identifying nanoparticles that are highly correlated with functional payload upon delivery. Additionally, the methods and compositions of the present invention enable measurement of function at cellular resolution, and applications across multiple species and disease models.

In particular, a method for screening nanoparticles of the present invention utilizes a payload mRNA encoding a protein of therapeutic relevance and containing a barcode to indicate biodistribution of the payload mRNA in vivo. The barcode on the payload mRNA is designed to correlate the chemical composition of the nanoparticle (e.g., lipid composition of a LNP) that encapsulates the payload mRNA. As such, the preferential in vivo distribution of nanoparticles in target tissues or cells can be determined based on the increased relative abundance of certain barcodes. As described herein, increased relative abundance of a barcode may be determined by fold above input (FAI) metric. Use of the FAI metric to determine payload mRNA is advantageous as it limits the impact of redundancy caused by high-throughput sequencing and does not require a unique molecular identifier (UMI) in each payload mRNA. Without being bounded by a particular theory, absence of UMI in the payload mRNA minimizes the disruption of the secondary structure of mRNA, improves stability of mRNA, and more closely represents a therapeutic mRNA design for clinical use.

In one aspect of the present invention, the method of in vivo nanoparticle screening for preferential delivery to a target tissue or cell comprises administering a plurality of nanoparticles having different lipid compositions to a non-human mammal, wherein individual nanoparticle encapsulates a payload mRNA comprising a barcode that correlates with the chemical composition of said individual nanoparticle; measuring relative abundance of each barcode in one or more target tissues or cells of interest; and comparing the relative abundance of each barcode to its corresponding relative input abundance to determine fold above input (FAI) for each barcode; wherein if the FAI of a barcode in a target tissue or cell is above a threshold, identifying the nanoparticle correlated with the barcode as a candidate nanoparticle suitable for preferential delivery to the target tissue or cell.

In some embodiments, the nanoparticles to be screened are lipid nanoparticles (LNPs).

In accordance with the present method, the relative abundance of the barcodes and payload mRNA encapsulated in the nanoparticles (such as LNPs) are determined and used for correlating the nanoparticle distribution. In some embodiments, the abundance of payload mRNA is measured by high throughput deep sequencing and next generation sequencing, single cell RNA sequencing (scRNA-seq), RT-qPCR and combinations thereof.

In some embodiments, the abundance of payload mRNA such as barcoded payload mRNA is measured by deep sequencing and/or next generation sequencing.

In some embodiments, the relative abundance of each barcode in target cells of interest is determined by single-cell RNA sequencing (scRNA-seq), wherein the relative abundance of each barcode and payload mRNA in target cells of interest is determined by assessing transcription profiles.

In some embodiments, the fold difference of the relative abundance of a barcoded payload mRNA is measured as FAI (Fold Above Input). In some examples, the FAI is determined by the following formula:

$$FAI = \frac{R e l ative abundance in organ}{Relative abundance {in}^{“} {Input}^{”}}$$

In some embodiments, the abundance of payload mRNA is further quantified by RT-qPCR. In some examples, the FAI is normalized according to the following formula:

$$log ( FAI ( x ) \cdot {2}^{dCTx} ) - log ( FAI ( y ) \cdot {2}^{dCTy} )$$

wherein FAI(x) is the FAI of a barcoded payload mRNA in Sample x; FAI(y) is the FAI(y) is the FAI of the barcoded payload mRNA in Sample y; dCTx is the difference of the cycle threshold (CT) values of a housekeeping gene and the barcoded payload mRNA in Sample x; and dCTy is the difference of the cycle threshold (CT) values of the housekeeping gene and the barcoded payload mRNA in Sample y.

In some embodiments, a pre-determined threshold is used; the threshold may set at above 2, or from 2-1000. In some embodiments, the threshold is 2, 3, 4, 5, 6, 8, 10, 12, 14, 16, 18, 20, 25, 30, 35, 40, 50, 60, 70, 80, 90, or 100. In other cases, the threshold may be determined by a control run in parallel with the screening with one or more control nanoparticle (e.g., LNPs).

In accordance with the present method, the payload mRNA comprises a RNA barcode in the 3′UTR region of the payload mRNA. The barcode may be contiguous or split into 2 or more segments. In some embodiments, the barcode comprises 4-30 nucleotides. In some embodiments, the barcode comprises 6-25 nucleotides. In some embodiments, the barcode comprises 8-20 nucleotides. As non-limiting examples, the barcode sequence comprises 6, 8, 10, 12, 18 or 20 nucleotides. In one embodiment, the barcode comprises 8 nucleotides. In another embodiment, the barcode comprises 20 nucleotides. In some embodiments, each barcode sequence has a hamming distance of 1, 2, 3, 4, or greater from any other barcode sequences. In one embodiment, each barcode sequence has a hamming distance of 3 or greater from any other barcode sequences.

In some embodiments, the barcode sequence has minimal predicted AG. In other embodiments, the barcode minimally impacts mRNA stability.

In some embodiments, the barcode is inserted into the 3′UTR sequence, or to the 5′ end of the 3′UTR sequence, or to the 3′ end of the 3′UTR sequence.

In some embodiments, the payload mRNA does not comprise a unique molecular identifier (UMI).

In some embodiments, the payload mRNA comprises the 3′ UTR from a mammalian gene. In some embodiments, the 3′UTR is from of mouse alpha-globin, human alpha globin 1, XBG, human beta globin, albumin or C3.

In some embodiments, the payload mRNA is about 3000 nucleotides to about 10,000 nucleotides in length, or about 4000 nucleotides to about 8000 nucleotides in length. In some examples, the payload mRNA comprises about 5000 nucleotides. In some embodiments, the payload mRNA comprises at least one modified nucleotide, and/or is codon optimized.

In some embodiments, the payload mRNA further comprises a microRNA binding site, wherein the microRNA binding site leads to accelerated mRNA degradation in specific cell-types. In some examples, the microRNA binding site binds miR-122, miR-126, miR-142, or miR-146. The microRNA-binding site is located in the 3′UTR region but does not overlap with the barcode. In some embodiments, the microRNA-binding site and the barcode have minimal or zero sequence homology.

In accordance with the present method, the in vivo nanoparticle screening platform may be used for screening lipid nanoparticles (LNPs) for preferential delivery to a target tissue or cell. In some embodiments, the LNP screening platform is used to screen LNP delivery in vivo in a non-human mammal. The non-human mammal is, but not limited to, a mouse, a rat, a rabbit, a pig, a goat, or a non-human primate.

In some embodiments, different tissues and cells are measured for LNP preferential delivery. In some embodiments, the target tissues of interest are selected from the group consisting of liver, spleen, bone marrow, lung, brain, heart, kidney, eye, lymph, muscle, spine, stomach, intestine, pancreas, and combination thereof. In some embodiments, the target cells of interest are selected from the group consisting of endothelial cells, fibroblasts, epithelial cells, neurons, glia cells, immune cells, hepatocytes, lipocytes, muscular cells, and combination thereof. In other embodiments, the target cells of interest are differentiated cells, progenitor cells, stem cells, and/or cancer cells.

In some embodiments, the relative abundance of each barcode in one or more tissues or cells is measured at 2 hours, 4 hours, 6 hours, 8 hours, 10 hours, 12 hours, 24 hours and/or 72 hours after administration. In some examples, the relative abundance of each barcode in one or more tissues or cells is measured at 2 hours and 24 hours after administration.

In accordance with the present method, a further validation of candidate LNPs from the initial screening is performed following the same process. In some embodiments, the method further comprises validating the candidate LNPs in a second non-human mammal, or in a biological system. In some examples, the second non-human mammal is a mouse, a rat, a rabbit, a pig, a goat, or a non-human primate. In one embodiment, the second non-human mammal is a different species from the screening. In some embodiments, the biological system is a cell-based system, or a disease model.

In accordance with the present method, the LNPs to be screened may further encapsulate a second RNA. In some embodiments, the second RNA is a guide RNA. In some embodiments, the payload mRNA and the second RNA is at a mass ratio from 1:4 to 4:1, e.g., 1:1, 1:2, 2:1, 1:3, 3:1, 1:4 and 4:1.

In accordance with the present method, the LNPs to be screened comprises at least one ionizable lipid, at least one helper lipid, at least one cholesterol-based and/or at least one PEGylated lipid.

In another aspect of the present invention, provided herein includes a payload mRNA comprising a barcode that encodes a polypeptide. In some embodiments, the payload mRNA encodes a nuclease, or variant thereof, wherein the nuclease is a nucleobase editor, or a member of CRISPR-associated protein family, or variant thereof. In one embodiment, the nuclease is a nucleobase editor, such as a cytidine base editor (CBE), an adenosine base editor (ABE), or variant thereof. In another embodiment, the nuclease is a CRISPR-associated protein, such as Cas1, Cas2, Cas3, Cas4, Cas5, Cas6, Cas6, Cas7, Cas8, Cas9, Cas10, Cas11, Cas12, Cas13, Cse1, Cse2, Csf1, Csm2, Csn2, Csx10, Csx11, Csy1, Csy2, Csy3, C2c1, C2c2, C2c3, C2c4, C2c5, C2c8, C2c9, Cpf1, or Cnr5, or variant thereof.

In yet another aspect, the present invention provides a method for producing a barcoded mRNA; the method comprises amplifying a linear protein coding nucleic acid sequence having a 3′UTR using a 5′ primer and a 3′ primer having a sequence specific to the 3′UTR and further comprising a nucleic acid barcode sequence, and transcribing in vitro the amplified linear protein coding nucleic acid sequence. In some embodiments, the 5′ primer is specific to a T7 promoter. In some examples, the 3′ primer comprises, in order from the 5′ to 3′ end, the sequence specific to the 3′UTR, the nucleic acid barcode, and a polyA tail. As non-limiting examples, the 3′ primer comprises the sequence of any one of SEQ ID NOs. 54-61.

In some embodiments, the linear protein coding nucleic acid sequence comprises, in order from the 5′ to 3′ end, a T7 promoter, a 5′UTR, an open reading frame, and the 3′UTR, and wherein the linear protein coding nucleic acid sequence does not comprise a nucleic acid barcode sequence.

### Definitions

In order for the present invention to be more readily understood, certain terms are first defined below. Additional definitions for the following terms and other terms are set forth throughout the specification.

A or An: The articles “a” and “an” are used herein to refer to one or to more than one (i.e., to at least one) of the grammatical object of the article. By way of example, “an element” means one element or more than one element.

Administer; As used herein, the terms “administer”, “administering” or “administration” include any method of delivery of a pharmaceutical composition or agent into a subject's system or to a particular region in or on a subject. In certain embodiments of the invention, the agent is administered topically. In certain embodiments of the invention, an agent is administered intravenously, intramuscularly, subcutaneously, intrathecally, intracerebral, intraventricular, intraspinal intradermally, intranasally, orally, transcutaneously, or mucosally.

Approximately or about: As used herein, the term “approximately” or “about,” as applied to one or more values of interest, refers to a value that is similar to a stated reference value. In certain embodiments, the term “approximately” or “about” refers to a range of values that fall within 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Associated with: Two events or entities are “associated” with one another, as that term is used herein, if the presence, level and/or form of one is correlated with that of the other. For example, a particular entity (e.g., polypeptide) is considered to be associated with a particular disease, disorder, or condition, if its presence, level and/or form correlates with incidence of and/or susceptibility to the disease, disorder, or condition (e.g., across a relevant population). In some embodiments, two or more entities are physically “associated” with one another if they interact, directly or indirectly, so that they are and remain in physical proximity with one another. In some embodiments, two or more entities that are physically associated with one another are covalently linked to one another; in some embodiments, two or more entities that are physically associated with one another are not covalently linked to one another but are non-covalently associated, for example by means of hydrogen bonds, van der Waals interaction, hydrophobic interactions, magnetism, and combinations thereof.

Barcode: As used herein, the term “barcode” generally refers to any label or identifier that can be used to convey information about an agent (e.g., a nucleic acid). A barcode can be a tag or a combination of tags attached to the agent. A barcode can be part of the agent, for example, an internal change to the agent or insertion to the agent. A barcode may be added to an agent reversibly or irreversibly. The barcode may be unique. Barcodes can have a variety of different formats, for example, barcodes can include: nucleic acid barcodes; random nucleic acid and/or amino acid sequences; modified nucleic acid barcodes, and synthetic nucleic acid and/or amino acid sequences. The phrases “barcode sequence” and “barcode”, as well as variations thereof, refer to an identifiable nucleotide sequence, such as an oligonucleotide or polynucleotide sequence. In the context of the present disclosure, a barcode is a short nucleic acid, ranging from 4-100, 4-80, 4-60, 4-50, 4-40, 4-30, 6-80, 6-60, 6-40, 8-30, or 8-20 nucleotides in length. A barcode can be a deoxyribonucleic acid (DNA) or ribonucleic acid (RNA) molecule. A barcode is 4 nucleotides, 5 nucleotides, 6 nucleotides, 7 nucleotides, 8 nucleotides, 9 nucleotides, 10 nucleotides, 11 nucleotides, 12 nucleotides, 13 nucleotides, 14 nucleotides, 15 nucleotides, 16 nucleotides, 17 nucleotides, 18 nucleotides, 19 nucleotides, 20 nucleotides, 21 nucleotides, 22 nucleotides, 23 nucleotides, 24 nucleotides, 25 nucleotides, or 30 nucleotides in length. The barcodes can allow for identification and/or quantification of individual sequencing-reads in real time. In some instances, barcodes employed are specially designed with specific unique (i.e., distinct) sequences that are significantly different from each other, even in the case of at least 1 or even 2 mutations.

Base Editor: By “base editor (BE),” or “nucleobase editor (NBE)” is meant an agent that binds a polynucleotide and has nucleobase modifying activity. In various embodiments, the base editor comprises a nucleobase modifying polypeptide (e.g., a deaminase) and a polynucleotide programmable nucleotide binding domain in conjunction with a guide polynucleotide (e.g., guide RNA). In various embodiments, the agent is a biomolecular complex comprising a protein domain having base editing activity, i.e., a domain capable of modifying a base (e.g., A, T, C, G, or U) within a nucleic acid molecule (e.g., DNA). In some embodiments, the polynucleotide programmable DNA binding domain is fused or linked to a deaminase domain. In one embodiment, the agent is a fusion protein comprising one or more domains having base editing activity. In another embodiment, the protein domains having base editing activity are linked to the guide RNA (e.g., via an RNA binding motif on the guide RNA and an RNA binding domain fused to the deaminase). In some embodiments, the domains having base editing activity are capable of deaminating a base within a nucleic acid molecule. In some embodiments, the base editor is capable of deaminating one or more bases within a DNA molecule. In some embodiments, the base editor is capable of deaminating a cytosine (C) or an adenosine (A) within DNA. In some embodiments, the base editor is capable of deaminating a cytosine (C) and an adenosine (A) within DNA. In some embodiments, the base editor is a cytidine base editor (CBE). In some embodiments, the base editor is an adenosine base editor (ABE). In some embodiments, the base editor is an adenosine base editor (ABE) and a cytidine base editor (CBE). In some embodiments, the base editor is a nuclease-inactive Cas9 (dCas9) fused to an adenosine deaminase. In some embodiments, the base editor is fused to an inhibitor of base excision repair, for example, a UGI domain, or a dISN domain. In some embodiments, the fusion protein comprises a Cas9 nickase fused to a deaminase and an inhibitor of base excision repair, such as a UGI or dISN domain. In other embodiments the base editor is an abasic base editor. Details of base editors are described in International PCT Application Nos. PCT/2017/045381 (WO2018/027078) and PCT/US2016/058344 (WO2017/070632), each of which is incorporated herein by reference for its entirety. Also see Komor, A. C., et al., “Programmable editing of a target base in genomic DNA without double-stranded DNA cleavage” Nature 533, 420-424 (2016); Gaudelli, N. M., et al., “Programmable base editing of A•T to G•C in genomic DNA without DNA cleavage” Nature 551, 464-471 (2017); Komor, A. C., et al., “Improved base excision repair inhibition and bacteriophage Mu Gam protein yields C:G-to-T:A base editors with higher efficiency and product purity” Science Advances 3:eaao4774 (2017), and Rees, H. A., et al., “Base editing: precision chemistry on the genome and transcriptome of living cells.” Nat Rev Genet. 2018 December; 19(12):770-788. doi: 10.1038/s41576-018-0059-1, the entire contents of which are hereby incorporated by reference.

Base Editing Activity: By “base editing activity” is meant acting to chemically alter a base within a polynucleotide. In one embodiment, a first base is converted to a second base. In one embodiment, the base editing activity is cytidine deaminase activity, e.g., converting target C•G to T•A. In another embodiment, the base editing activity is adenosine or adenine deaminase activity, e.g., converting A•T to G•C. In another embodiment, the base editing activity is cytosine or cytidine deaminase activity, e.g., converting target C•G to T•A and adenosine or adenine deaminase activity, e.g., converting A•T to G•C.

Base Editor System: The term “base editor system” refers to a system for editing a nucleobase of a target nucleotide sequence. In various embodiments, the base editor (BE) system comprises (1) a polynucleotide programmable nucleotide binding domain (e.g., Cas9), a deaminase domain and a cytidine deaminase domain for deaminating nucleobases in the target nucleotide sequence; and (2) one or more guide polynucleotides (e.g., guide RNA) in conjunction with the polynucleotide programmable nucleotide binding domain. In various embodiments, the base editor (BE) system comprises a nucleobase editor domains selected from an adenosine deaminase or a cytidine deaminase, and a domain having nucleic acid sequence specific binding activity. In some embodiments, the base editor system comprises (1) a base editor (BE) comprising a polynucleotide programmable DNA binding domain and a deaminase domain for deaminating one or more nucleobases in a target nucleotide sequence; and (2) one or more guide RNAs in conjunction with the polynucleotide programmable DNA binding domain. In some embodiments, the polynucleotide programmable nucleotide binding domain is a polynucleotide programmable DNA binding domain. In some embodiments, the base editor is a cytidine base editor (CBE). In some embodiments, the base editor is an adenine or adenosine base editor (ABE). In some embodiments, the base editor is an adenine or adenosine base editor (ABE) or a cytidine base editor (CBE).

In some embodiments, a polynucleotide programmable nucleotide binding domain can target a deaminase domain to a target nucleotide sequence by non-covalently interacting with or associating with the deaminase domain. For example, in some embodiments, the nucleobase editing component, e.g., the deaminase component can comprise an additional heterologous portion or domain that is capable of interacting with, associating with, or capable of forming a complex with an additional heterologous portion or domain that is part of a polynucleotide programmable nucleotide binding domain. In some embodiments, the additional heterologous portion may be capable of binding to, interacting with, associating with, or forming a complex with a polypeptide. In some embodiments, the additional heterologous portion may be capable of binding to, interacting with, associating with, or forming a complex with a polynucleotide. In some embodiments, the additional heterologous portion may be capable of binding to a guide polynucleotide. In some embodiments, the additional heterologous portion may be capable of binding to a polypeptide linker. In some embodiments, the additional heterologous portion may be capable of binding to a polynucleotide linker. The additional heterologous portion may be a protein domain. In some embodiments, the additional heterologous portion may be a K Homology (KH) domain, a MS2 coat protein domain, a PP7 coat protein domain, a SfMu Com coat protein domain, a steril alpha motif, a telomerase Ku binding motif and Ku protein, a telomerase Sm7 binding motif and Sm7 protein, or an RNA recognition motif.

Biologically active: As used herein, the phrase “biologically active” refers to a characteristic of any agent that has activity in a biological system, and particularly in an organism. For instance, an agent that, when administered to an organism, has a biological effect on that organism, is considered to be biologically active. In particular embodiments, where a peptide is biologically active, a portion of that peptide that shares at least one biological activity of the peptide is typically referred to as a “biologically active” portion.

Complementary: As used herein, complementary refers to a nucleic acid strand that forms Watson-Crick base pairing, such that A base pairs with T, and C base pairs with G, or non-traditional base pairing with bases on a second nucleic acid strand. In other words, it refers to nucleic acids that hybridize with each other under appropriate conditions.

Clustered Interspaced Short Palindromic Repeat (CRISPR)-associated (Cas) system: As used herein, CRISPR-Cas9 system refers to nucleic acids and/or proteins involved in the expression of, or directing the activity of, CRISPR-effectors, including sequences encoding CRISPR effectors, RNA guides, and other sequences and transcripts from a CRISPR locus. In some embodiments, the CRISPR system is an engineered, non-naturally occurring CRISPR system. In some embodiments, the components of a CRISPR system may include a nucleic acid(s) (e.g., a vector) encoding one or more components of the system, a component(s) in protein form, or a combination thereof.

CRISPR-associatedprotein (Cas): The term “CRISPR-associated protein,” “CRISPR effector,” “effector,” or “CRISPR enzyme” as used herein refers to a protein that carries out an enzymatic activity or that binds to a target site on a nucleic acid specified by a RNA guide. In different embodiments, a CRISPR effector has endonuclease activity, nickase activity, exonuclease activity, transposase activity, and/or excision activity.

crRNA: The term “CRISPR RNA” or “crRNA,” as used herein, refers to a RNA molecule including a guide sequence used by a CRISPR effector to target a specific nucleic acid sequence. Typically, crRNAs contain a sequence that mediates target recognition and a sequence that forms a duplex with a tracrRNA. In some embodiments, the crRNA: tracrRNA duplex binds to a CRISPR effector.

Ex Vivo: As used herein, the term “ex vivo” refers to events that occur in cells or tissues, grown outside rather than within a multi-cellular organism.

Half-Life: As used herein, the term “half-life” is the time required for a quantity such as protein concentration or activity to fall to half of its value as measured at the beginning of a time period.

Hamming Distance: As used herein, the term “hamming distance” is a metric (in the mathematical sense) used in error correction theory to measure the distance between two codewords. A hamming distance between two barcodes measures the level of differences between the nucleotide sequences of the barcode. Generally speaking, a hamming distance between two barcodes reflects the minimal number of changes that needs to be made to convert one barcode to the other. For example, a hamming distance between barcode “TTCTCTGC” and barcode “TTCACTAC” is 2. A hamming distance between barcode “ATGTCGCT” and barcode “TCTTCGCT” is 3. A hamming distance between barcode “ATCGTTGG” and barcode “ATCTCGTG” is 4.

Homology: The term “homology” when used in relation to nucleic acids refers to a degree of complementarity. There may be partial homology (i.e., partial identity) or complete homology (i.e., complete identity).

Improve, increase, or reduce: As used herein, the terms “improve,” “increase” or “reduce,” or grammatical equivalents, indicate values that are relative to a baseline measurement, such as a measurement in the same individual prior to initiation of the treatment described herein, or a measurement in a control subject (or multiple control subject) in the absence of the treatment described herein. A “control subject” is a subject afflicted with the same form of disease as the subject being treated, who is about the same age as the subject being treated.

Indel: As used herein, the term “indel” refers to insertion or deletion of bases in a nucleic acid sequence. It commonly results in mutations and is a common form of genetic variation.

In Vitro: As used herein, the term “in vitro” refers to events that occur in an artificial environment, e.g., in a test tube or reaction vessel, in cell culture, etc., rather than within a multi-cellular organism.

In Vivo: As used herein, the term “in vivo” refers to events that occur within a multi-cellular organism, such as a human and a non-human animal. In the context of cell-based systems, the term may be used to refer to events that occur within a living cell (as opposed to, for example, in vitro systems).

mRNA: As used herein, the term “mRNA” refers to messenger RNA which is a polynucleotide that encodes at least one polypeptide.

microRNA binding site: As used herein, the term “microRNA binding site” refers to a nucleotide sequence which is complementary or partially complementary to at least a portion of a microRNA. The binding site sequence can be a perfect match, meaning that the binding site sequence has perfect complementarity to the microRNA. Alternatively, the binding site sequence can be partially complementary, meaning that one or more mismatches may occur when the microRNA is base paired to the binding site.

Mutation: As used herein, the term “mutation” has the ordinary meaning in the art, and includes, for example, point mutations, substitutions, insertions, deletions, inversions, and deletions.

Nucleoside: As used herein, the term “nucleoside” refers to a molecule having a purine or pyrimidine base covalently linked to a ribose or deoxyribose sugar. Exemplary nucleosides include adenosine, guanosine, cytidine, uridine and thymidine. The term “nucleotide” refers to a nucleoside having one or more phosphate groups joined in ester linkages to the sugar moiety. Exemplary nucleotides include nucleoside monophosphates, diphosphates and triphosphates. The terms “polynucleotide,” “oligonucleotide” and “nucleic acid molecule” are used interchangeably herein and refer to a polymer of nucleotides, either deoxyribonucleotides or ribonucleotides, of any length joined together by a phosphodiester linkage between 5′ and 3′ carbon atoms. Oligonucleotide generally is between about 5 and about 100 nucleotides of single- or double-stranded DNA. The terms also refer to both double- and single-stranded molecules. Unless otherwise specified or required, any embodiment of this invention that comprises a polynucleotide encompasses both the double-stranded form and each of two complementary single-stranded forms known or predicted to make up the double-stranded form. A polynucleotide is composed of a specific sequence of four nucleotide bases: adenine (A); cytosine (C); guanine (G); thymine (T); and uracil (U) for thymine when the polynucleotide is RNA. The terms “RNA,” “RNA molecule” and “ribonucleic acid molecule” refer to a polymer of ribonucleotides. The terms “DNA,” “DNA molecule” and “deoxyribonucleic acid molecule” refer to a polymer of deoxyribonucleotides. DNA and RNA can be synthesized naturally (e.g., by DNA replication or transcription of DNA, respectively). RNA can be post-transcriptionally modified. DNA and RNA can also be chemically synthesized. DNA and RNA can be single-stranded (i.e., ssRNA and ssDNA, respectively) or multi-stranded (e.g., double stranded, i.e., dsRNA and dsDNA, respectively). “mRNA” or “messenger RNA” is single-stranded RNA that specifies the amino acid sequence of one or more polypeptide chains. As used herein, the term “isolated RNA” (e.g., “isolated mRNA”) refers to RNA molecules which are substantially free of other cellular material, or culture medium when produced by recombinant techniques, or substantially free of chemical precursors or other chemicals when chemically synthesized.

Payload: As used herein, the term “payload” refers to any molecule and collection of molecules formulated by a delivery vehicle. The payload molecule can be a nucleic acid molecule (e.g., RNA and DNA), a protein (such as an antibody or fusion protein), a peptide, a lipid, a small molecule or the like, or combinations thereof. In some embodiments, the payload is a RNA (e.g., mRNA, guide RNA, siRNA, microRNA, etc.) encapsulated by a nanoparticle such as lipid nanoparticle. In certain embodiments, the payload molecule is an mRNA, such as an mRNA comprising a barcode.

Polypeptide: The term “polypeptide” as used herein refers to a sequential chain of amino acids linked together via peptide bonds. The term is used to refer to an amino acid chain of any length, but one of ordinary skill in the art will understand that the term is not limited to lengthy chains and can refer to a minimal chain comprising two amino acids linked together via a peptide bond. As is known to those skilled in the art, polypeptides may be processed and/or modified. As used herein, the terms “polypeptide” and “peptide” are used inter-changeably.

Protein: The term “protein” as used herein refers to one or more polypeptides that function as a discrete unit. If a single polypeptide is the discrete functioning unit and does not require permanent or temporary physical association with other polypeptides in order to form the discrete functioning unit, the terms “polypeptide” and “protein” may be used interchangeably. If the discrete functional unit is comprised of more than one polypeptide that physically associate with one another, the term “protein” refers to the multiple polypeptides that are physically coupled and function together as the discrete unit.

RNA guide: The term RNA guide refers to an RNA molecule that facilitates the targeting of a protein described herein to a target nucleic acid. Exemplary “RNA guides” or “guide RNAs” or “gRNA” include, but are not limited to, crRNAs or crRNAs in combination with cognate tracrRNAs. The latter may be independent RNAs or fused as a single RNA using a linker (sgRNAs). In some embodiments, the RNA guide is engineered to include a chemical or biochemical modification, in some embodiments, an RNA guide may include one or more nucleotides.

Single cell: As used herein, a “single cell” refers to one cell. Single cells useful in the methods described herein can be obtained from a tissue of interest, or from a biopsy, blood sample, or cell culture. Additionally, cells from specific organs, tissues, tumors, neoplasms, or the like can be obtained and used in the methods described herein.

Subject: The term “subject”, as used herein, means any subject for whom diagnosis, prognosis, or therapy is desired. For example, a subject can be a mammal, e.g., a human or non-human primate (such as an ape, monkey, orangutan, or chimpanzee), a dog, cat, guinea pig, rabbit, rat, mouse, horse, cattle, or cow.

sgRNA: The term “sgRNA” or “single guide RNA” refers to a single guide RNA containing (i) a guide sequence (crRNA sequence) and (ii) a Cas9 nuclease-recruiting sequence (tracrRNA).

Substantial identity: The phrase “substantial identity” is used herein to refer to a comparison between amino acid or nucleic acid sequences. As will be appreciated by those of ordinary skill in the art, two sequences are generally considered to be “substantially identical” if they contain identical residues in corresponding positions. As is well known in this art, amino acid or nucleic acid sequences may be compared using any of a variety of algorithms, including those available in commercial computer programs such as BLASTN for nucleotide sequences and BLASTP, gapped BLAST, and PSI-BLAST for amino acid sequences. Exemplary such programs are described in Altschul, et al., Basic local alignment search tool, J. Mol. Biol., 215(3): 403-410, 1990; Altschul, et al., Methods in Enzymology; Altschul et al., Nucleic Acids Res. 25:3389-3402, 1997; Baxevanis et al., Bioinformatics: A Practical Guide to the Analysis of Genes and Proteins, Wiley, 1998; and Misener, et al., (eds.), Bioinformatics Methods and Protocols (Methods in Molecular Biology, Vol. 132), Humana Press, 1999. In addition to identifying identical sequences, the programs mentioned above typically provide an indication of the degree of identity. In some embodiments, two sequences are considered to be substantially identical if at least 50%, 55%, 60%, 65%, 70%, 75%, 80%, 85%, 90%, 91%, 92%, 93%, 94%, 95%, 96%, 97%, 98%, 99% or more of their corresponding residues are identical over a relevant stretch of residues. In some embodiments, the relevant stretch is a complete sequence. In some embodiments, the relevant stretch is at least 10, 15, 20, 25, 30, 35, 40, 45, 50, 55, 60, 65, 70, 75, 80, 85, 90, 95, 100, 125, 150, 175, 200, 225, 250, 275, 300, 325, 350, 375, 400, 425, 450, 475, 500 or more residues.

Target Nucleic Acid: The term “target nucleic acid” as used herein refers to nucleotides of any length (oligonucleotides or polynucleotides) to which the CRISPR-Cas9 system binds, either deoxyribonucleotides, ribonucleotides, or analogs thereof. Target nucleic acids may have three-dimensional structure, may including coding or non-coding regions, may include exons, introns, mRNA, tRNA, rRNA, siRNA, shRNA, miRNA, ribozymes, cDNA, plasmids, vectors, exogenous sequences, endogenous sequences. A target nucleic acid can comprise modified nucleotides, include methylated nucleotides, or nucleotide analogs. A target nucleic acid may be interspersed with non-nucleic acid components. A target nucleic acid is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases.

tracrRNA: The term “tracrRNA” or “trans-activating crRNA” as used herein refers to an RNA including a sequence that forms a structure required for a CRlSPR-associated protein to bind to a specified target nucleic acid.

### BRIEF DESCRIPTION OF THE DRAWING

Drawings are for illustration purposes only; not for limitation.

FIG. 1 is a presentative diagram depicting barcoded mRNA design and nanoparticle screening using the payload mRNA comprising a barcode.

FIG. 2 is a flow-chart showing production of a payload mRNA comprising a barcode using PCR generated template.

FIG. 3 demonstrates the editing function of three barcoded mRNAs encoding an editing enzyme.

FIG. 4 is an illustration of sequencing to measure barcodes and payload mRNA abundance.

FIG. 5A illustrates the experimental flow and FIG. 5B is a plot showing the normalized FAI of each barcode in liver, spleen and bone marrow at 2 hours and 24 hours after administration.

FIG. 6A shows the barcoded payload mRNA biodistribution of pooled 4 LNPs in spleen.

FIG. 6B shows the barcoded payload mRNA biodistribution of pooled 4 LNPs in bone marrow.

FIG. 6C shows the barcoded payload mRNA biodistribution of pooled 4 LNPs in liver.

FIG. 7 is a flow of RT-qPCR assay for measuring payload mRNA comprising a barcode.

FIG. 8A shows direct comparisons between organs (liver and spleen) using RT-qPCR normalization.

FIG. 8B shows direct comparisons between LNPs across different time points and organs using RT-qPCR normalization.

FIG. 8C shows direct comparisons between LNPs across different doses and organs using RT-qPCR normalization.

### DETAILED DESCRIPTION

Various aspects of the invention are described in detail in the following sections. The use of sections is not meant to limit the invention. Each section can apply to any aspect of the invention. In this application, the use of “or” means “and/or” unless stated otherwise.

The present invention relates to compositions and methods for in vivo screening nanoparticles (e. g., lipid nanoparticles) for preferential delivery to a target tissue or cell. The tissue or cell tropism of a lipid nanoparticle is characterized using the present compositions and methods. The present nanoparticle screening methods allow screening multiple nanoparticles with increased sensitivity and accuracy. This delivery screening platform, using payload mRNAs that are barcoded, encapsulated in nanoparticles, can accelerate in vivo screening and the design of nanoparticles for mRNA-based therapy.

The present in vivo screening platform can be used to screen any nanoparticles that are suitable for in vivo delivery of therapeutic agents. In particular, methods described herein can be used to screen any lipid nanoparticles. The methods described herein for screening lipid nanoparticles (LNPs) can be easily modified to screen any other nanoparticles and other delivery vehicles.

The in vivo nanoparticle, such as LNP screening systems and methods of the present invention use barcoded payload mRNAs and an optimized assay to measure barcodes and payload mRNAs; thereby screen LNPs that can deliver mRNAs to a target tissue or cell. Use of barcoded mRNAs (payload of LNPs) eliminates use of additional barcodes or other molecular identifiers that may affect LNP in vivo delivery. The optimized assay for measuring relative abundance of payload mRNA increases the sensitivity of the present methods. In general, a library of lipid nanoparticles with different lipid compositions encapsulates barcoded mRNAs as payloads, in which each barcode correlates with the lipid composition of a LNP. By direct measuring the barcode counts of the mRNA payloads, the relative abundance of each barcode (i.e., payload mRNA) in a target tissue or cell of interest will identify the tropism of its corresponding LNP to that tissue or cell; thereby providing a direct correlation between the payload (e.g., barcoded mRNA encoding a protein) and LNP. As compared with other in vivo LNP screening approaches, the approaches discussed herein enhance the correlation of an mRNA payload and the delivery vehicle, nanoparticle (i.e., lipid nanoparticle). More importantly, the present approaches improve and simplify barcode measurement (e.g., without using UMI), and increase the accuracy by quantification of the payload (e.g., barcoded mRNA), for example, using the FAI and RT-qPCR.

In addition, the present payload mRNA based LNP screening methods allow measurement of efficacy of LNP delivery to a target cell at cellular resolution, for example, by single cell RNA sequencing (scRNA-seq).

Provided in the present disclosure also include compositions, e.g., mRNA comprising a barcode, compositions of mRNA comprising a barcode and a second RNA molecule, which find use in the present screening methods.

### Methods of In Vivo Screening LNPs for Preferential Tissue and Cell Distribution

The present invention relates to, among other things, methods for in vivo screening lipid nanoparticles (LNPs) for preferential delivery to a target tissue or cell. The tissue or cell tropism of a lipid nanoparticle for mRNA delivery is directly characterized by measuring the abundance of payload mRNA comprising a barcode in a target tissue or cell, rather than the encapsulation of additional barcodes and/or molecular identifier that may potentially alter LNP structure and subsequent in vivo delivery.

As a non-limiting example, the method for screening lipid nanoparticles (LNPs) for preferential delivery to a target tissue or cell includes administering a plurality of LNPs having different lipid compositions to a non-human mammal, wherein individual LNP encapsulates a payload mRNA comprising a barcode that correlates with the lipid composition of said individual LNP; measuring relative abundance of each barcode in one or more target tissues or cells of interest; comparing the relative abundance of each barcode to its corresponding relative input abundance to determine fold above input (FAI) for each barcode; and if the FAI of a barcode in a target tissue or cell is above a threshold, identifying the LNP correlated with the barcode as a candidate LNP suitable for preferential delivery to the target tissue or cell.

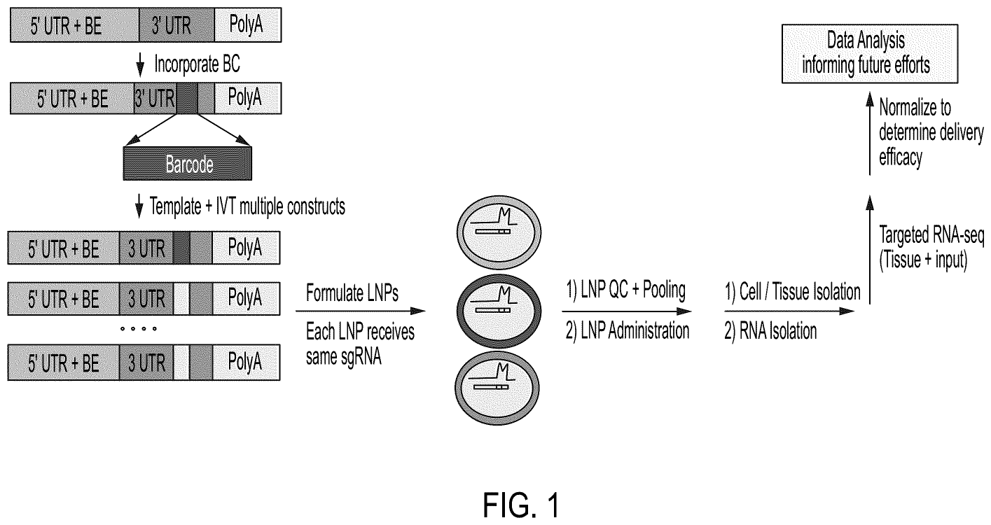

FIG. 1 is a representative diagram depicting the present in vivo delivery platform consisting of a library of LNPs that encapsulate a plurality of barcoded mRNAs for LNP distributions in different tissues and cells.

In some embodiments, the method uses payload mRNA comprising a barcode. A barcoded payload mRNA is an mRNA molecule comprising a barcode sequence in the 3′UTR region of the mRNA. The barcoded payload mRNAs employed in the present screening methods are described herein below.

In some embodiments, the relative abundance of each barcode (e.g., mRNA encapsulated in a nanoparticle, such as a LNP) is measured and used to correlate with the particle's tissue or cell distribution.

As used herein, the term “relative abundance” refers to the frequency of a barcode (i.e. mRNA) in a sample, e.g., percentage of barcode sequence counts. The sample is an input sample, i.e., a library of LNPs encapsulating barcoded payload mRNAs before administration. The relative abundance of a barcode in the input can be used to normalize the relative abundance of the barcode in a target tissue or cell after administration of the input LNPs. As discussed herein, the sample is a target tissue or cell isolated from a non-human mammal after the LNP administration. The relative abundance of a barcode (i.e., mRNA) in a target tissue or cell is measured and normalized with its relative abundance in the input sample. The fold differences (e.g., FAI) in the relative abundance levels indicate the LNP containing the payload mRNA distribution in different tissues and/or cells.

The fold difference threshold of different barcoded payload mRNAs in a target tissue of cell may be determined to be from 2-1000. In some embodiments, the fold difference threshold may be determined to be 2 or more, 3 or more, 4 or more, 5 or more, 6 or more, 7 or more, 8 or more, 9 or more, 10 or more, 12 or more, 14 or more, 16 or more, 18 or more, 20 or more, 30 or more, 40 or more, 50 or more, 60 or more, 70 or more, 80 or more, 90 or more, 100 or more, 150 or more, 200 or more, 250 or more, 300 or more, or 500 or more. In some embodiments, the fold difference is determined by a pre-determined threshold. The threshold is from 2 to 1000, from 2 to 500, from 2-200, from 2-100, from 5-500, from 5-100, from 10-100, or from 5-50. In some embodiments, the threshold is 2, 4, 6, 8, 10, 12, 16, 18, 20, 25, 30, 35, 40, 50, 60, 70, 80, 90 or 100. In some embodiments, the threshold is determined by a control run in parallel with the nanoparticle screening assay. One or more control LNPs can be used in parallel with the screening to determine the threshold.

The relative abundance of payload mRNA can be measured and determined by deep-sequencing and high throughput next-generation sequencing. In some embodiments, the barcoded payload mRNA profile and relative abundance in a single cell can be measured by single-cell RNA sequencing (scRNA-seq). The single cell transcript profile can be used to identify a LNP that is preferentially delivered to this cell type, providing the LNP preferential delivery at cellular solution.

In some embodiments, the relative abundance of a barcoded payload mRNA can be determined using RT-qPCR, alone or in combination with other methods such as deep-sequencing.

Detailed descriptions of methods for determining relative abundances of barcoded payload m RNA s are discussed below.

As a non-limiting example, the fold difference of the relative abundance of a barcode (i.e., payload mRNA) is measured as FAI (Fold Above Input) below:

$$FAI = \frac{R e l ative abundance in organ}{Relative abundance {in}^{“} {Input}^{”}}$$

In one embodiment, the fold difference of the relative abundance of a payload mRNA is determined using the formula:

$$log ( FAI ( x ) \cdot {2}^{dCTx} ) - log ( FAI ( y ) \cdot {2}^{dCTy} )$$

wherein FAI(x) is the FAI of a barcoded payload mRNA in Sample x; FAI(y) is the FAI(y) is the FAI of the barcoded payload mRNA in Sample y; dCTx is the difference of the cycle threshold (CT) values of a housekeeping gene and the barcoded payload mRNA in Sample x; and dCTy is the difference of the cycle threshold (CT) values of the housekeeping gene and the barcoded payload mRNA in Sample y.

In some embodiments, the non-human mammal is a mouse (e.g., a mouse with different genetic background), a rat, a rabbit, a pig, a goat, or a NHP (non-human primate). In some embodiments, the non-human mammal is a disease model such as a mouse model for a disease.

In accordance with the present invention, the nanoparticle such as LNP screening methods optimize the quantification assay for barcode sequences and mRNAs by measuring relative abundance of barcoded payload mRNAs and use of the same to determine LNP distribution. Accordingly, the payload mRNA comprises only the barcode sequence in the 3′ UTR region. The payload mRNA does not comprise a secondary barcode, e.g., Unique Molecular Identifier (UMI) which is generally included in nucleic acid sample library for reducing PCR and sequencing variation.

### Multiplex LNPs Screening

The present screening method can potentially allow for several thousand unique barcoded-mRNA LNP formulations to be administered into a non-human mammal and screened for preferential delivery. In some embodiments, a plurality of LNPs encapsulates a plurality of barcoded payload mRNAs; each individual LNP encapsulates an individual barcoded payload mRNA comprising a barcode that correlates with the lipid composition of said individual LNP. The plurality of barcoded mRNA-LNPs is pooled and injected to a non-human mammal for delivery screening.

In some embodiments, the plurality of LNPs may comprise over hundreds of LNPs with different lipid compositions. The number of LNPs can be screened in an assay may range from 10 to more than a thousand LNPs. The number of LNPs in an input sample may include 10-1000, 20-1000, 50-1000, 100-1000, 200-1000, 300-1000, 500-1000, 200-800, 500-800, 400-600, or 100-500 different LNPs. In some embodiments, the number of mRNAs that can be multiplexed will determine the pool LNPs to be screened with the present method.

In some embodiments, the plurality of LNPs is administered to a non-human mammal for in vivo screening of preferential delivery of LNPs to one or more target tissues or cells.

In accordance with the present invention, the relative input abundance of each barcode in the LNP input may be pre-determined (e.g., with a defined percentage in the input sample); alternatively, the input abundance may be measuring barcode sequence counts using deep-sequencing/NGS. The relative abundance of each barcode is then measured in a target tissue or cell after administration of the pooled input. The fold difference of each barcode in each tissue or cell will correlate with the distribution of the LNP encapsulating the barcode in that tissue or cell. As non-limiting examples, if the fold difference of the relative abundance of a barcode in the liver is above the threshold (e.g., 5), it indicates that the LNP containing the payload mRNA is enriched in the liver, which is then identified as candidate LNP for liver mRNA delivery. By multiplexing screen, a pool of LNPs can be screened for their preferential distribution in different organs, tissues and cells simultaneously.

In accordance with the present invention, multiple nanoparticles may be simultaneously screened following the methods described herein.

### Multiplex Tissues and Cells

The present in vivo LNP screening method can be used to screen the preferential delivery of LNPs to one or more organs, tissues and cells.

In some embodiments, a target organ or tissue (as used herein, the terms “organ” and “tissue” are used interchangeably) includes but is not limited to liver, spleen, bone marrow, brain, eye, muscle, lymph, spine, pancreas, heart, lung, stomach, intestine, kidney, and combination thereof.

Accordingly, the preferential delivery of a LNP can be measured at cellular level. The target cells may include but are not limited to, endothelial cells, fibroblasts, epithelial cells, neurons, glia cells, immune cells, hepatocytes, lipocytes, muscular cells, or combinations thereof. In some embodiments, the cells may include but are not limited to differentiated cells, progenitor cells, stem cells, and/or cancer cells.

After a defined period of time post-administration, one or more tissues or cells are isolated and processed for extracting total mRNAs. The total mRNA can be extracted from one or more tissues or cells using any commercially available kits. The barcode sequence counts in the mRNA sample can be measured by deep sequencing/NGS. In some embodiments, the sample is a single cell. The RNA profile including the barcoded payload mRNA delivered to the single cell can be performed by single cell PCR sequencing (scRNA-seq). In some embodiments, RT-qPCR may also be used in combination with scRNA-seq.

Comparison of the barcoded payload mRNA profiles in different cell types can identify the cell tropism of a LNP at cellular solution.

### Multiplex Time Points Screening

Another aspect of the present screening method is that use of fold difference of relative abundance of each barcode against its relative abundance in the input sample (pre-administration sample) can be employed to measure and compare LNP distributions in a target tissue or cell at different time points.

In some embodiments, the tissue and cell samples are isolated and analyzed at 2 hours, 4 hours, 6 hours, 8 hours, 10 hours, 12 hours, 24 hours, 48 hours, and 72 hours post-administration. In some embodiments, the tissue and cell samples are isolated and analyzed at 2 hours and 24 hours after administration.

### Screening Across Species and Validation of Candidate LNPs

Another advantage of the present method is that the method can be used to compare the preferential delivery of LNPs to a target tissue or cell across the species, using the same relative abundance determination of payload mRNAs with barcodes.

In some embodiments, the method further comprises the steps of validating candidate LNPs that are identified with preferential delivery to a target tissue or cell (e.g., to liver or hepatocyte). The candidate LNPs may be further screened and validated in a second non-human mammal following the same process in the initial screening. The FAI and normalized quantification of the barcodes and payloads mRNAs in the target tissue or cell may be used to further validate the tissue or cell preference of the candidate LNP, to establish a more trustful correlation of the barcode abundance and LNP distribution in the target tissue or cell.

In some embodiments, the second non-human mammal is the same mammal used for the initial LNP screening.

In other embodiments, the second non-human mammal is different from the mammal in the initial LNP screening.

The second non-human mammal may be a mouse, a rat, a rabbit, a pig, a goat, or a non-human primate.

In some embodiments, the system for candidate LNP validation is a cell-based system or a disease model (e.g., a mouse model for a disease).

Relative Abundance Assay and Correlation with LNP Bio-Distribution

When practicing the present nanoparticles, such as LNPs, screening methods, the relative abundance of the barcode from a barcoded payload mRNA is measured for correlating such barcode relative abundance with LNP's preferential delivery to a target tissue or cell. The relative abundance of a barcoded payload mRNA can be determined by sequencing barcode counts using deep-sequencing and high-throughput next generation sequencing (NGS), single cell RNA sequencing (scRNA-seq) technology, and combination thereof. RT-qPCR is also used to normalize the relative abundance.

### High Throughput Sequencing

In some embodiments, the in vivo LNP delivery in a target tissue or cell is quantified via high throughput deep sequencing. The payload mRNA with a barcoded region in its 3′ untranslated region (UTR) can be quantified directly using deep-sequencing technologies (as referred as high-throughput deep sequencing or next generation sequencing (NGS). The abundance and quantification of barcode sequence counts in a target tissue or cell sample indicate the LNP delivery to that tissue or cell.

The terms “deep sequencing” and “ultra-deep sequencing” are used interchangeably herein and refer to approaches that use massively parallel sequencing technologies to obtain large numbers of sequences corresponding to relatively short, targeted sequences. A targeted sequence is the barcode sequence. In some embodiments, the barcode sequence counts and payload mRNAs are sequenced allowing identification and quantification for the abundance of the barcodes. A single sequencing primer for sequencing the barcode and barcode-specific primer in a single read may be used.

Next generation sequencing (NGS) may be used to measure the abundance of payload mRNA when practicing the present method. Next-generation sequencing (NGS) is a massively parallel sequencing technology that offers ultra-high throughput, scalability, and speed. The technology is used to determine the order of nucleotides, for example in the targeted regions of RNA. NGS provides a depth of the payload mRNA information in the target tissues and cells. With NGS based RNA sequencing, the payload mRNA delivered to a target tissue or cell can be deeply sequenced and quantified; the quantification of the payload mRNA in the target tissue or cell will provide in depth of LNP preferential delivery to that tissue or cell.

As a non-limiting example, a pool of LNPs that encapsulate barcoded payload mRNAs are administered to a non-human mammal. The LNP delivery of barcoded payload mRNA is quantified by high-throughput deep sequencing (e.g., using any suitable sequencing platform such as those commercially available from Illumina, Life Technologies, or the like) or NGS. The relative abundance of a barcode (i.e., a payload mRNA) in a target tissue or cell is measured and normalized with its relative abundance in the input sample before administration. The fold differences (e.g., FAI) in the relative abundance levels indicate the LNP containing the payload mRNA distribution in different tissues and/or cells.

In some embodiments, different doses of barcoded payload mRNAs for LNP formulation may be used for LNP delivery screening. Low doses of total barcoded payload mRNAs can still be detected using deep sequencing. The present nanoparticle screening platform can detect nanoparticle doses at low doses using deep sequencing. The doses may range from 0.0001 mg/kg to 100 mg/kg. In some embodiments, the doses are from about 0.0001 mg/kg to 0.1 mg/kg, or from about 0.001 mg/kg to abut 100 mg/kg, or from 0.001 mg/kg to about 10 mg/kg, or from about 0.1 mg/kg to about 100 mg/kg, or from 0.1 mg/kg to about 10 mg/kg, or from 0.1 mg/kg to about 1 mg/kg.

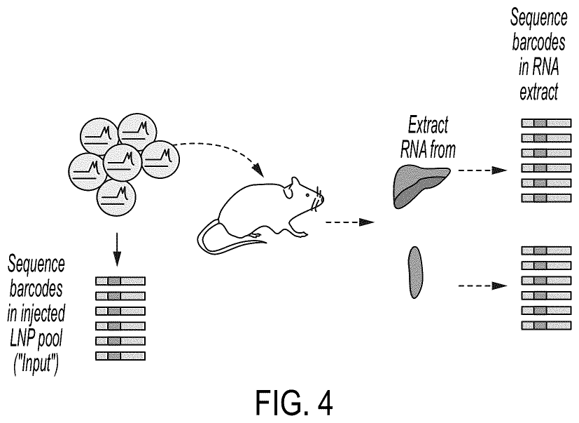

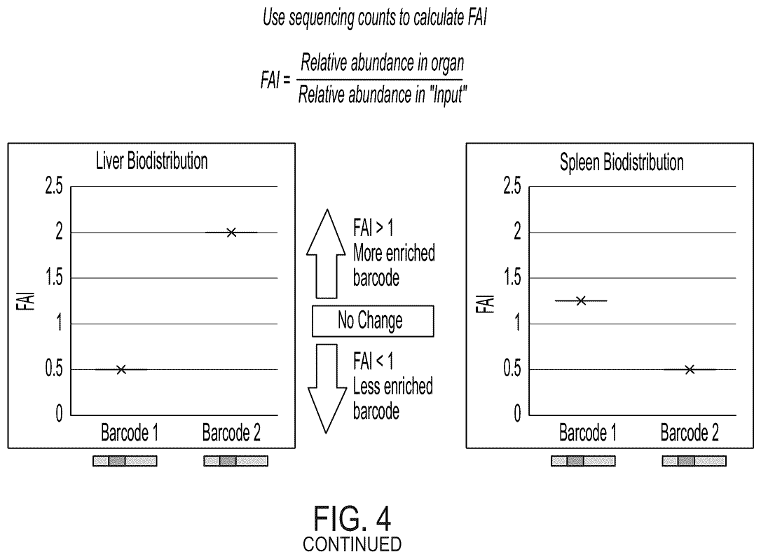

As a non-limiting example, upon sequencing the barcode counts, the relative abundance is measured as Fold Above Input (FAI). As used herein, the term “Fold Above Input (FAI)” is the normalized relative abundance of a barcode in a selected sample as compared to its frequency in the input. The FAI of a barcode indicates how a LNP's abundance changes relative to the rest of the LNP pool. The FAI value of a RNA barcode is calculated by normalizing the relative abundance in the RNA barcode sequence counts in the isolated samples to its relative abundance in the administration input. For example, the value ‘1’ represents an LNP appearing at the same frequency in the isolated sample as it does in the administration pool, representing that it displays neutral tropism to the cell-type measured relative to other LNP populations in that same administration pool. The FAI then indicates the performance of an LNP relative to the input LN P composition, FIG. 4 shows the flowchart for calculating FA. The following formula is used for calculating FAI:

$$FAI = \frac{R e l ative abundance in organ}{Relative abundance {in}^{“} {Input}^{”}}$$

### Single-Cell RNA-Seq

In some embodiments, single-cell RNA sequencing (scRNA-seq) is used to profile the barcoded payload mRNAs at single-cell resolution. The method includes generating mRNA profiles from a target cell to obtain the single cell mRNA data, including barcoded payload mRNAs from the target cell. The payload mRNA profiles then can be used to characterize the LNP delivery preferential to the target cell.

In accordance with the present invention, single cell transcript profiling after administration of payload mRNA-loaded LNPs is obtained using scRNA-seq technology. By single cell RNA profiling, which combines isolation of single cell (e.g., using FACS) and high-throughput next generation sequencing techniques, the present method can detect and quantitate RNA abundance of all or substantially all of mRNAs including payload mRNAs delivered into the single cell, such that a single cell RNA profile on a transcriptomic-wide scale is obtained. The RNA profiling can provide at a single cell level, the LNP profiling with preferential delivery to the target cell.

Single cell can be isolated with a commonly used technique, such as cell sorting (FACS) technology and microfluidic technology. In some embodiments, a biomarker associated with a cell type is used to identify the cell type in scRNA-seq.

In some embodiments, the single cell RNA profile of a target cell is obtained at different time points, e.g., 2 hours, 4 hours, 6 hours, 8 hours, 12 hours, 24 hours and 72 hours, after administration of barcoded payload mRNA-loaded LNPs. Comparison of the single cell RNA profiles of the same cell at different time points can correlate the payload mRNA abundance with LNP distribution over the time.

In some embodiments, the single cell RNA profiles of different cell types are obtained. Comparison of the RNA profiles of different cell types can correlate the payload mRNA abundance with LNPs across cell types.

In some embodiments, the single cell RNA profiles for different doses of payload mRNA are obtained. Comparison the RNA profiles of different mRNA doses can correlate the payload mRNA abundance with LNP delivery across doses.

### Multiplex RT-gPCR Assay

As discussed above, a multiplex quantitative PCT (RT-qPCR) is developed and may be employed to generate payload mRNA profile data in a target tissue or cell after the barcoded payload mRNA-LNP administration. The RT-qPCR assay may be used when practicing the present in vivo nanoparticle screening methods. The RT-qPCR assay is capable accurate quantitation of the total mRNAs extracted from a target tissue or cell including barcoded mRNAs that have been delivered to the target tissue or cell. Using the same primers targeting the coding sequence of the barcoded payload mRNA and 3′UTR, all the payload mRNAs in the sample can be amplified in a high multiplex reaction, reducing variations between different reactions for individual barcoded mRNA. Each pre-amplified payload mRNA is quantitated via quantitative PCR (qPCR) method. The quantification of each barcoded mRNA in a tissue or cell sample indicates the quantitative distribution of the LNP encapsulating the payload mRNA.

In some embodiments, the relative abundance of the mRNA payloads from a target tissue or cell is determined by RT-PCR.

In some embodiments, to avoid the sample preparation variation, e.g., mRNA extraction variation, a housekeeping gene in the sample (e.g., ActB, GAPDH, AHSA1, PTEN, SSB, and TBP) is used as “normalization transcript”. As used herein, these housekeeping genes have expression levels (i.e., RNA counts) that are relatively constant among different samples.

In some embodiments, the RT-qPCR data can normalize the FAI in cases that the percentage of a LNP (correlating to its barcode) appears higher in abundance despite no actual changes in abundance. The normalization using RT-qPCR data further increases the sensitivity and accuracy of the present screening methods. In this context, the FAI of a payload mRNA is multiplied by the linearized RT-qPCR data (2^{dCT}) In some embodiments, the following formula is used to normalize the FAI using RT-qPCR data of the barcoded payload mRNA, thereby converting back to quantitative data (relative to a housekeeping gene).

$$log ( FAI ( x ) \cdot {2}^{dCTx} ) - log ( FAI ( y ) \cdot {2}^{dCTy} )$$

wherein FAI(x) is the FAI of a barcoded payload mRNA in Sample x; FAI(y) is the FAI(y) is the FAI of the barcoded payload mRNA in Sample y; dCTx is the difference of the cycle threshold (CT) values of a housekeeping gene (e.g., ActB) and the barcoded payload mRNA in Sample x; and dCTy is the difference of the cycle threshold (CT) values of the housekeeping gene and the barcoded payload mRNA in Sample y.

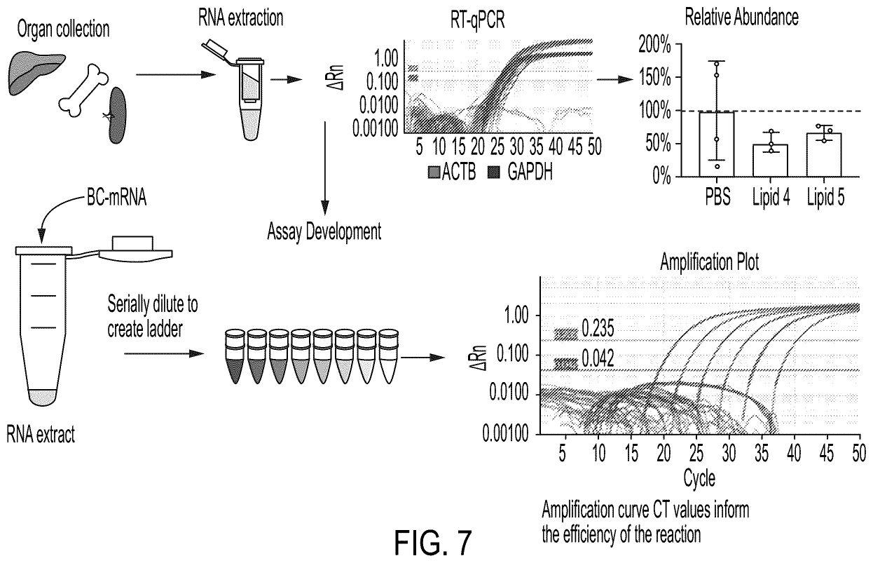

As a general process, approximately the same amount of tissue (e.g., 10-20 mg) is extracted from RNAlater-preserved samples. The barcoded payload mRNAs and a housekeeping gene (e.g., ActB) in the sample are quantitated in a multiplex RT-qPCR. The relative amount of barcodes (“bc-mRNA”) to actB transcripts is represented by the difference in their cycle threshold (“Ct” or “CT”) values: 2^{(CtactB-Ctbc-mRNA)}. Assuming the housekeeping gene actB is expressed similarly in all tissues and cells being compared, Ct_{actB}-Ct_{bc-mRNA} can be thought of as representing the approximate number of barcodes per cell. Differences in actB signals are interpreted are differences in extraction yields. The same housekeeping gene across validation for different species (e.g., mouse, rat and non-human primate) is used to keep the results consistent. An exemplary RT-qPCR assay to quantitatively measure barcoded payload mRNAs according to one embodiment of the present disclosure is schematically illustrated in FIG. 7.

By using RT-qPCR quantifications of both the barcode and a housekeeping gene, a comparison across tissues, time, dose, species and screens can be performed.

Design of Payload mRNA

The present invention uses payload mRNA for in vivo screening of lipid nanoparticles for their preferential delivery to various tissues and cells. In some embodiments, the payload mRNA is designed to comprise a barcode sequence. A barcoded mRNA according to the present invention is an mRNA molecule comprising a short barcode sequence wherein the barcode is integrated into the nucleotide sequence of the mRNA. Preferentially the barcode locates in the untranslated regions of the mRNA.

### Barcode

In accordance with the present invention, the barcode is a nucleic acid barcode, e.g., a RNA barcode.

A barcode comprises a short nucleotide sequence. In some embodiments, the barcode comprises about 4-30 nucleotides. In some embodiments, the barcode comprises about 6-25 nucleotides. In some embodiments, the barcode comprises about 6-20 nucleotides. In some embodiments, the barcode comprises about 6-15 nucleotides. In some embodiments, the barcode comprises about 6-10 nucleotides. In some embodiments, the barcode is about 4, 5, 6, 7, 8, 9, 10, 11, 12, 13, 14, 15, 16, 17, 18, 19, 20, 21, 22, 23, 24, or 25 nucleotides in length or longer. In some embodiments, the length of a barcode is about 4, 5, 6, 7, 8, 9, 10, 11, 12, 13, 14, 15, 16, 17, 18, 19, 20, 21, 22, 23, 24, or 25 nucleotides in length or shorter. In some embodiments, the barcode is at least 4 nucleotides in length. In some embodiments, the barcode is at least 5 nucleotides in length. In some embodiments, the barcode is at least 6 nucleotides in length. In some embodiments, the barcode is at least 7 nucleotides in length. In some embodiments, the barcode is at least 8 nucleotides in length. In some embodiments, the barcode is at least 9 nucleotides in length. In some embodiments, the barcode is at least 10 nucleotides in length. In some embodiments, the barcode is at least 11 nucleotides in length. In some embodiments, the barcode is at least 12 nucleotides in length. In some embodiments, the barcode is at least 13 nucleotides in length. In some embodiments, the barcode is at least 14 nucleotides in length. In some embodiments, the barcode is at least 15 nucleotides in length. In some embodiments, the barcode is at least 16 nucleotides in length. In some embodiments, the barcode is at least 17 nucleotides in length. In some embodiments, the barcode is at least 18 nucleotides in length. In some embodiments, the barcode is at least 19 nucleotides in length. In some embodiments, the barcode is at least 20 nucleotides in length. In some embodiments, the barcode is at least 21 nucleotides in length. In some embodiments, the barcode is at least 22 nucleotides in length. In some embodiments, the barcode is at least 23 nucleotides in length. In some embodiments, the barcode is at least 24 nucleotides in length. In some embodiments, the barcode is at least 25 nucleotides in length. In some embodiments, the barcode is at least 26 nucleotides in length. In some embodiments, the barcode is at least 27 nucleotides in length. In some embodiments, the barcode is at least 28 nucleotides in length. In some embodiments, the barcode is at least 29 nucleotides in length. In some embodiments, the barcode is at least 30 nucleotides in length.

In one embodiment, the barcode comprises 8 nucleotides.

In another embodiment, the barcode comprises 20 nucleotides.

The barcode sequence does not interfere with the barcoded mRNA stability or expression. The barcode sequence may be designed by computation to optimize the GC content and secondary structure (e.g., to minimize AG). Generally speaking, any two suitable barcodes according to the invention have a hamming distance of at least 1. As non-limiting example, if a barcode has N nucleotides (i.e., 4 nucleotides, 5 nucleotides, 6 nucleotides . . . 30 nucleotides), any two suitable barcodes or a collection of barcodes have a hamming distance of 1 to N (i.e., 1, 2, 3, 4, 5, 6, 8 . . . N-5, N-4, N-3, N-2, N-1 and N). In some embodiments, if a suitable barcode has 8 nucleotides, any two suitable barcodes or a collection of barcodes have a hamming distance of at least 1, or a hamming distance of at least 2, or a hamming distance of at least 3, or a hamming distance of at least 4, or a hamming distance of at least 5, or a hamming distance of at least 6, or a hamming distance of at least 7, or a hamming distance of at least 8. In some embodiments, any suitable barcodes according to the invention can be a mix of one of the above.

In some embodiments, the barcode sequence is contiguous. In some embodiments, the barcode is non-contiguous. A non-contiguous barcode can be a barcode with its nucleotide sequence interrupted with one or more constant nucleotide sequence of the mRNA. In some embodiments, a non-contiguous barcode is present in the 3′ UTR region and with its nucleotide sequence interrupted with one or more sequences from 3′ UTR. In some embodiments, a non-contiguous barcode has at least a portion of its nucleotide sequence present outside the 3′ UTR region. In some embodiments, a non-contiguous barcode has nucleotide sequences present at different locations throughout the length of the mRNA.

In some embodiments, the barcode comprises unmodified nucleotides. In some embodiments, the barcode comprises modified nucleotides. In some embodiments, the barcode comprises a combination of unmodified and modified nucleotides.

In some embodiments, the barcode is inserted within the untranslated region of a payload mRNA to produce a barcoded payload mRNA. In some embodiments, the barcode is inserted within the 3′UTR region of the payload mRNA. In some embodiments, the barcode is located at the 5′ end of the start nucleobase of the 3′UTR of the payload mRNA. In some embodiments, the barcode locates at the 3′ end of the stop nucleobase of the 3′ UTR of the payload mRNA.

### 3′ Untranslated Regions (3′UTR)

A barcode may locate at the 3′ untranslated region (3′ UTR) of a mRNA. As used herein, 3′UTR is the sequence segment of a mRNA that immediately follows the translation stop codon.

In some embodiments, the payload mRNA contains a 3′UTR derived from any mammalian gene. In some embodiments, the 3′UTR is derived from a mouse mRNA, or a rat mRNA, a non-human primate (NHP) mRNA, or a human mRNA.

The 3′UTR may be the 3′UTR from a naturally isolated mRNA, or a synthetic nucleotide sequence processed from nucleic acid synthesizers commonly used in the art, or a nucleotide sequence genetically engineered according to known techniques. A “complete” or “entire” naturally-occurring 3′UTR sequence usually starts after the coding sequence 3′UTR stop codon (i.e., just after the open reading frame) and ends with the 3′UTR terminal nucleobase. In some embodiments, the 3′UTR comprises the 3′UTR sequence from a naturally occurring mRNA. In some embodiments, the 3′UTR comprises a synthetic 3′UTR sequence. In some embodiments, the 3′UTR sequence has at least 70% identity to a naturally occurring 3′UTR sequence. In some embodiments, the 3′UTR sequence has at least 75% identity to a naturally occurring 3′UTR sequence. In some embodiments, the 3′UTR sequence has at least 80% identity to a naturally occurring 3′UTR sequence. In some embodiments, the 3′UTR sequence has at least 85% identity to a naturally occurring 3′UTR sequence. In some embodiments, the 3′UTR sequence has at least 90% identity to a naturally occurring 3′UTR sequence. In some embodiments, the 3′UTR sequence has at least 91% identity to a naturally occurring 3′UTR sequence. In some embodiments, the 3′UTR sequence has at least 92% identity to a naturally occurring 3′UTR sequence. In some embodiments, the 3′UTR sequence has at least 93% identity to a naturally occurring 3′UTR sequence. In some embodiments, the 3′UTR sequence has at least 94% identity to a naturally occurring 3′UTR sequence. In some embodiments, the 3′UTR sequence has at least 95% identity to a naturally occurring 3′UTR sequence. In some embodiments, the 3′UTR sequence has at least 96% identity to a naturally occurring 3′UTR sequence. In some embodiments, the 3′UTR sequence has at least 97% identity to a naturally occurring 3′UTR sequence. In some embodiments, the 3′UTR sequence has at least 98% identity to a naturally occurring 3′UTR sequence. In some embodiments, the 3′UTR sequence has at least 99% identity to a naturally occurring 3′UTR sequence.

A 3′UTR varies in size. On average, the 3′UTR is about 50-1500 nucleotides in length. In some embodiments, the 3′UTR used in the present invention comprises about 50-1200 nucleotides, 50-1000 nucleotides, 50-800 nucleotides, 100-800 nucleotides, 200-800 nucleotides, 500-1200 nucleotides, or 500-800 nucleotides. In some embodiments, the 3′UTR comprises about 200 nucleotides. In some embodiments, the 3′UTR comprises about 200 nucleotides. In some embodiments, the 3′UTR comprises about 250 nucleotides. In some embodiments, the 3′UTR comprises about 300 nucleotides. In some embodiments, the 3′UTR comprises about 350 nucleotides. In some embodiments, the 3′UTR comprises about 400 nucleotides. In some embodiments, the 3′UTR comprises about 450 nucleotides. In some embodiments, the 3′UTR comprises about 500 nucleotides. In some embodiments, the 3′UTR comprises about 550 nucleotides. In some embodiments, the 3′UTR comprises about 600 nucleotides. In some embodiments, the 3′UTR comprises about 650 nucleotides. In some embodiments, the 3′UTR comprises about 700 nucleotides. In some embodiments, the 3′UTR comprises about 750 nucleotides. In some embodiments, the 3′UTR comprises about 800 nucleotides. In some embodiments, the 3′UTR comprises about 850 nucleotides. In some embodiments, the 3′UTR comprises about 900 nucleotides. In some embodiments, the 3′UTR comprises about 950 nucleotides. In some embodiments, the 3′UTR comprises about 1000 nucleotides. In some embodiments, the 3′UTR comprises about 1200 nucleotides. In some embodiments, the 3′UTR comprises about 1500 nucleotides.

In some embodiments, the 3′UTR used in the present invention comprises unmodified nucleotides. In some embodiments, the 3′UTR used in the present invention comprises modified nucleotides. In some embodiments, the 3′UTR used in the present invention comprises a combination of unmodified and modified nucleotides.

In some embodiments, the barcode of the present invention may be inserted into the 3′UTR. In some embodiments, the barcode is inserted at a position in the 3′UTR sequence that does not interfere with the structure and function of the 3′UTR sequence.

In some embodiments, the barcode of the present invention may locate upstream (i.e., the 5′ end) of the 3′UTR. As non-limiting examples, the barcode is placed after the stop codon of the payload mRNA.

In some embodiments, the barcode of the present invention may locate downstream (i.e., the 3′ end) of the 3′UTR. As non-limiting examples, the barcode is placed at the junction of 3′UTR and poly(A) tail.

In accordance with the present invention, the 3′UTR comprising a barcode is presented by a sequence pattern (from 5′ to 3′) (sequence of the 5′ portion of the 3′UTR)—(barcode sequence)-(sequence of the 3′ portion of 3′UTR)

In one embodiment, the 3′UTR comprises the sequence of 5′ TTAATTAAGCTGCCTTCTGCGGGGCTTGCCTTCTGGCCATGCCCTTCTTCTCTCCCTTGCACCT GTACCTCTTG(NNNNNNNN) GTCTTTGAATAAAGCCTGAGTAGGAAG-3′ (SEQ ID NO. 1), wherein each of N is A, T, G or C.

In another embodiment, the 3′UTR comprises the sequence of 5′ TTAATTAAGCTGCCTTCTGCGGGGCTTGCCTTCTGGCCATGCCCTTCTTCTCTCCCTTGCACCT GTACCTCTTG(NNNNNNNN NNNNNNNNNNNN) GTCTTTGAATAAAGCCTGAGTAGGAAG-3′ (SEQ ID NO. 2), wherein each of N is A, T, G or C.

An exemplary non-contiguous barcode that locates in different regions of a 3′UTR may be presented by a sequence pattern as follows (from 5′ to 3′):
(sequence of the 5′ portion of the 3′UTR)—(barcode segment 1)-(sequence of 3′UTR)-(barcode segment 2)—(sequence of the 3′ portion of 3′UTR).

In some embodiments, the 3′UTR is derived from mouse alpha-globin, human alpha-globin, XBG, human beta globin and C3.

microRNA Binding Sites

In some embodiments, the barcoded payload mRNA may further comprise one or more microRNA binding sites. The microRNA binding sites are located in the 3′UTR region of a barcoded payload mRNA but may also be located in other regions of the barcoded payload mRNA. The microRNA binding site comprises a nucleotide sequence which is complementary or partially complementary, to at least a portion of a microRNA. In some embodiment, the microRNA binding site is 75%, 80%, 85%, 90%, 91%, 92% 93%, 94%, 95%, 96%, 97%, 98%, or 99% complementary to the wild type binding site of a microRNA. Partially complementary binding sites preferably contain perfect or near perfect complementarity to the seed region of the microRNA. As used herein, the term “seed region” of a microRNA binding site consists of the 5′ region of the microRNA from about nucleotide 2 to about nucleotide 8.

In some embodiments, the microRNA binding sites comprise wild type microRNA binding site sequences, or mutated microRNA binding site sequences. The mutations in the microRNA binding sites do not disrupt inhibition by endogenous microRNAs.

A microRNA binding site sequence may comprise about 4-30 nucleotides. In some embodiments, the microRNA binding site comprises about 6 nucleotides. In some embodiments, the microRNA binding site comprises about 7 nucleotides. In some embodiments, the microRNA binding site comprises about 8 nucleotides. In some embodiments, the microRNA binding site comprises about 9 nucleotides. In some embodiments, the microRNA binding site comprises about 10 nucleotides. In some embodiments, the microRNA binding site comprises about 11 nucleotides. In some embodiments, the microRNA binding site comprises about 12 nucleotides. In some embodiments, the microRNA binding site comprises about 13 nucleotides. In some embodiments, the microRNA binding site comprises about 14 nucleotides. In some embodiments, the microRNA binding site comprises about 15 nucleotides. In some embodiments, the microRNA binding site comprises about 16 nucleotides. In some embodiments, the microRNA binding site comprises about 17 nucleotides. In some embodiments, the microRNA binding site comprises about 18 nucleotides. In some embodiments, the microRNA binding site comprises about 19 nucleotides. In some embodiments, the microRNA binding site comprises about 20 nucleotides. In some embodiments, the microRNA binding site comprises about 21 nucleotides. In some embodiments, the microRNA binding site comprises about 22 nucleotides. In some embodiments, the microRNA binding site comprises about 23 nucleotides. In some embodiments, the microRNA binding site comprises about 24 nucleotides. In some embodiments, the microRNA binding site comprises about 25 nucleotides. In some embodiments, the microRNA binding site comprises about 26 nucleotides. In some embodiments, the microRNA binding site comprises about 27 nucleotides. In some embodiments, the microRNA binding site comprises about 28 nucleotides. In some embodiments, the microRNA binding site comprises about 29 nucleotides. In some embodiments, the microRNA binding site comprises about 30 nucleotides.

In some embodiments, a specific microRNA binding site is placed in the 3′UTR of a payload mRNA. The expression and stability of the payload mRNA becomes sensitive to the microRNA phenotype of the cell-type to which the LNP encapsulating the payload mRNA is delivered, An example of a miRNA that is specifically present in a given cell type is miR-122, which is normally present in high levels in animal liver cells. Delivery of a LNP encapsulating a barcoded payload mRNA containing a miR-122 site to a normal liver cell would result in repression of the barcoded payload mRNA in that cell. In contrast, delivery of the same LNP to a cell that does not express miR-122, would result in expression of the barcoded mRNA, in that cell. In this way, a target cell is selectively targeted for the payload mRNA delivery and expression, by selecting a miRNA binding site that corresponds to a microRNA that is not expressed in the target cell, but is expressed in surrounding cells.

In some embodiments, the payload mRNA comprises a miRNA-126 binding site which binds miRNA-126 that is one of the most abundantly expressed miRNAs in endothelial cells. In some embodiments, the microRNA binding site binds miR-142, In some embodiments, the microRNA binding binds miR-146.

In some embodiments, the microRNA binding site in the 3′UTR region of the payload mRNA is separately from the barcode sequence. In some embodiments, the microRNA binding site and the barcode have minimal or zero sequence homology. In some examples, the microRNA binding site does not interfere with the barcode measurement and vice versa.

### Unique Molecular Identifier (UMI)

In some embodiments, the payload mRNA does not comprise a UMI (Unique Molecular Identifier). As used herein, a UMI is a short nucleic acid sequence that provides error correction and increase accuracy during sequencing. The UMI is used to uniquely tag each molecule in a library. According to the present disclosure, measurement of the abundance of barcodes and normalization of the barcode quantification make is unnecessary to use UMIs to reduce PCR and sequencing variation and errors. It is an advantage to eliminate the introduction of another short sequence into the 3′UTR, which may affect the 3′UTR structure and function, thereby affecting the barcoded payload mRNA, e.g., the stability of the barcoded payload mRNA.

In accordance, the present invention also provides a method for screening lipid nanoparticles (LNPs) for preferential delivery to a target tissue or cell, comprising: (i) administering a plurality of LNPs having different lipid compositions to a non-human mammal, wherein individual LNP encapsulates a payload mRNA comprising a barcode that correlates with the lipid composition of said individual LNP, and wherein the barcode containing payload mRNA does not comprise a unique molecular identifier (UMI); and (ii) determining relative abundance of each barcode in one or more target tissues or cells of interest as compared to a reference, thereby identifying a candidate LNP suitable for preferential delivery to a target tissue or cell.

In some embodiments, the present invention provides a method for producing a barcoded mRNA; the method comprises amplifying a linear protein coding nucleic acid sequence having a 3′UTR using a 5′ primer and a 3′ primer having a sequence specific to the 3′UTR and further comprising a nucleic acid barcode sequence, and transcribing in vitro the amplified linear protein coding nucleic acid sequence. In some embodiments, the 5′ primer is specific to a T7 promoter. In some examples, the 3′ primer comprises, in order from the 5′ to 3′ end, the sequence specific to the 3′UTR, the nucleic acid barcode, and a polyA tail. As non-limiting example, the 3′ primer comprises the sequence of any one of SEQ ID NOs. 54-61.

In some embodiments, the linear protein coding nucleic acid sequence comprises, in order from the 5′ to 3′ end, a T7 promoter, a 5′UTR, an open reading frame, and the 3′UTR, and wherein the linear protein coding nucleic acid sequence does not comprise a nucleic acid barcode sequence.

In some embodiments, the linear protein coding nucleic acid sequence is produced from a plasmid.

Proteins Encoded by Payload mRNA

As discussed above, provided by the present disclosure also include payload mRNAs comprising barcodes. The barcoded payload mRNA as disclosed herein comprises a barcode sequence in the untranslated region, preferentially at the 3′ untranslated region (3′UTR). The present payload mRNA encodes a polynucleotide, a protein, or variant thereof that provides a measurable signal upon delivery in vivo. In some embodiments, the payload mRNA encodes a peptide. In some embodiments, the payload mRNA encodes a protein, or variant thereof. In some embodiments, the payload mRNA encodes an enzyme, or variant thereof. In some embodiments, the payload mRNA encodes a protein hormone, or variant thereof. In some embodiments, the payload mRNA encodes an antibody, or variant thereof. In some embodiments, the payload mRNA encodes a structural protein, or variant thereof. In some embodiments, the payload mRNA encodes a nuclease, or variant thereof.

The payload mRNA can be in various sizes. In some embodiments, the payload mRNA has a size from about 3000-10,000 nucleotides, or about 5,000-10,000 nucleotides, or about 4,500-6,000 nucleotides. In some embodiments, the payload mRNA comprises about 3,000 nucleotides. In some embodiments, the payload mRNA comprises about 4,000 nucleotides. In some embodiments, the payload mRNA comprises about 4,500 nucleotides. In some embodiments, the payload mRNA comprises about 5,000 nucleotides. In some embodiments, the payload mRNA comprises about 10,000 nucleotides.

### CRISPR and Base Editors

In some embodiments, a payload mRNA encodes a nuclease or variant thereof.

In some embodiments, the nuclease may include a Cas protein (also called a “Cas nuclease”) from a CRISPR/Cas system. In some embodiments, payload mRNA encodes a Cas protein from a CRISPR/Cas system. The Cas protein may comprise at least one domain that interacts with a guide RNA (gRNA). Additionally, the Cas protein may be directed to a target sequence by a guide RNA. The guide RNA interacts with the Cas protein as well as the target sequence such that, once directed to the target sequence, the Cas protein is capable of cleaving the target sequence. In certain embodiments, e.g., Cas9, the Cas protein is a single-protein effector, an RNA-guided nuclease. In some embodiments, the guide RNA provides the specificity for the targeted cleavage, and the Cas protein may be universal and paired with different guide RNAs to cleave different target sequences. The terms Cas protein and Cas nuclease are used interchangeably herein.

In some embodiments, payload mRNA encodes a Type-I, Type-II, or Type-III system component. Updated classification schemes for CRISPR/Cas loci define Class 1 and Class 2 CRISPR/Cas systems, having Types I to V or VI. See, e.g., Makarova et al., Nat Rev Microbiol, 13(11): 722-36 (2015); Shmakov et al., Molecular Cell, 60:385-397 (2015). Class 2 CRISPR/Cas systems have single protein effectors. Cas proteins of Types II, V, and VI may be single-protein, RNA-guided endonucleases, herein called “Class 2 Cas nucleases.” Class 2 Cas nucleases include, for example, Cas9, Cpf1, C2c1, C2c2, and C2c3 proteins. Cpf1 protein, Zetsche et al., Cell, 163: 1-13 (2015), is homologous to Cas9, and contains a RuvC-like nuclease domain. Cpf1 sequences of Zetsche are incorporated by reference in their entirety. See, e.g., Zetsche, Tables 51 and S3. Accordingly, in some embodiments, payload mRNA encodes a Cas9, Cpf1, C2c1, C2c2, or C2c3 protein.

In some embodiments, the Cas protein may be from a Type-II CRISPR/Cas system, i.e., a Cas9 protein from a CRISPR/Cas9 system. In some embodiments, the Cas protein may be from a Class 2 CRISPR/Cas system, i.e., a single-protein Cas nuclease such as a Cas9 protein or a Cpf1 protein. The Cas9 and Cpf1 family of proteins are enzymes with DNA endonuclease activity, and they can be directed to cleave a desired nucleic acid target by designing an appropriate guide RNA.

A Type-II CRISPR/Cas system component may be from a Type-IIA, Type-IIB, or Type-IIC system. Cas9 and its orthologs are encompassed. In some embodiments, payload mRNA encodes a Cas9 from Streptococcus pyogenes, Streptococcus thermophilus, Streptococcus sp., Staphylococcus aureus, Listeria innocua, Lactobacillus gasseri, Francisella novicida, Wolinella succinogenes, Sutterella wadsworthensis, Gamma proteobacterium, Neisseria meningitidis, Campylobacter jejuni, Pasteurella multocida, Fibrobacter succinogene, Rhodospirillum rubrum, Nocardiopsis dassonvillei, Streptomyces pristinaespiralis, Streptomyces viridochromogenes, Streptomyces viridochromogenes, Streptosporangium roseum, Streptosporangium roseum, Alicyclobacillus acidocaldarius, Bacillus pseudomycoides, Bacillus selenitireducens, Exiguobacterium sibiricum, Lactobacillus delbrueckii, Lactobacillus salivarius, Lactobacillus buchneri, Treponema denticola, Microscilla marina, Burkholderiales bacterium, Polaromonas naphthalenivorans, Polaromonas sp., Crocosphaera watsonii, Cyanothece sp., Microcystis aeruginosa, Synechococcus sp., Acetohalobium arabaticum, Ammonifex degensii, Caldicelulosiruptor becscii, Candidatus Desulforudis, Clostridium botulinum, Clostridium difficile, Finegoldia magna, Natranaerobius thermophilus, Pelotomaculum thermopropionicum, Acidithiobacillus caldus, Acidithiobacillus ferrooxidans, Allochromatium vinosum, Marinobacter sp., Nitrosococcus halophilus, Nitrosococcus watsoni, Pseudoalteromonas haloplanktis, Ktedonobacter racemifer, Methanohalobium evestigatum, Anabaena variabilis, Nodularia spumigena, Nostoc sp., Arthrospira maxima, Arthrospira platensis, Arthrospira sp., Lyngbya sp., Microcoleus chthonoplastes, Oscillatoria sp., Petrotoga mobilis, Thermosipho africanus, Streptococcus pasteurianus, Neisseria cinerea, Campylobacter lari, Parvibaculum lavamentivorans, Corynebacterium diphtheria, or Acaryochloris marina. In some embodiments, payload mRNA encodes a Cas9 protein from Streptococcus pyogenes. In some embodiments, payload mRNA encodes a Cas9 protein from Streptococcus thermophilus. In some embodiments, payload mRNA encodes a Cas9 protein from Neisseria meningitidis. In some embodiments, payload mRNA encodes a Cas9 protein from Staphylococcus aureus.

In some embodiments, payload mRNA encodes a Cas protein that comprises more than one nuclease domain. For example, a Cas9 protein may comprise at least one RuvC-like nuclease domain (e.g., Cpf1) and at least one HNH-like nuclease domain (e.g., Cas9). In some embodiments, the Cas9 protein may be capable of introducing a DSB in the target sequence. In some embodiments, the Cas9 protein may be modified to contain only one functional nuclease domain. For example, the Cas9 protein may be modified such that one of the nuclease domains is mutated or fully or partially deleted to reduce its nucleic acid cleavage activity. In some embodiments, the Cas9 protein may be modified to contain no functional RuvC-like nuclease domain. In other embodiments, the Cas9 protein may be modified to contain no functional HNH-like nuclease domain. In some embodiments in which only one of the nuclease domains is functional, the Cas9 protein may be a nickase that is capable of introducing a single-stranded break (a “nick”) into the target sequence. In some embodiments, a conserved amino acid within a Cas9 protein nuclease domain is substituted to reduce or alter a nuclease activity. In some embodiments, the Cas protein nickase may comprise an amino acid substitution in the RuvC-like nuclease domain. Exemplary amino acid substitutions in the RuvC-like nuclease domain include D10A (based on the S. pyogenes Cas9 protein). In some embodiments, payload mRNA encodes a nickase. In some embodiments, a nickase may comprise an amino acid substitution in the HNH-like nuclease domain. Exemplary amino acid substitutions in the HNH-like nuclease domain include E762A, H840A, N863A, H983A, and D986A (based on the S. pyogenes Cas9 protein). In some embodiments, the nuclease system described herein may comprise a nickase and a pair of guide RNAs that are complementary to the sense and antisense strands of the target sequence, respectively. The guide RNAs may direct the nickase to target and introduce a DSB by generating a nick on opposite strands of the target sequence (i.e., double nicking). Chimeric Cas9 proteins may also be used, where one domain or region of the protein is replaced by a portion of a different protein. For example, a Cas9 nuclease domain may be replaced with a domain from a different nuclease such as Fok1. A Cas9 protein may be a modified nuclease.

As non-limiting examples, a payload mRNA encodes a CRISPR-associated protein selected from Cas1, Cas2, Cas3, Cas4, Cas5, Cas6, Cas6, Cas7, Cas8, Cas9, Cas10, Cas11, Cas12, Cas13, Cse1, Cse2, Csf1, Csm2, Csn2, Csx10, Csx11, Csy1, Csy2, Csy3, C2c1, C2c2, C2c3, C2c4, C2c5. C2c8, C2c9, Cpf1, and Cmr5.

In some embodiments, payload mRNAs encode nucleobase editors that edit, modify or alter a target nucleotide sequence of a polynucleotide. Nucleobase editors described herein typically include a polynucleotide programmable nucleotide binding domain and a nucleobase editing domain (e.g., adenosine deaminase or cytidine deaminase). A polynucleotide programmable nucleotide binding domain, when in conjunction with a bound guide polynucleotide (e.g., gRNA), can specifically bind to a target polynucleotide sequence and thereby localize the base editor to the target nucleic acid sequence desired to be edited.

In certain embodiments, the nucleobase editors comprise one or more features that improve base editing activity. For example, any of the nucleobase editors may comprise a Cas9 domain that has reduced nuclease activity. In some embodiments, any of the nucleobase editors provided herein may have a Cas9 domain that does not have nuclease activity (dCas9), or a Cas9 domain that cuts one strand of a duplexed DNA molecule, referred to as a Cas9 nickase (nCas9). Without wishing to be bound by any particular theory, the presence of the catalytic residue (e.g., H840) maintains the activity of the Cas9 to cleave the non-edited (e.g., non-deaminated) strand opposite the targeted nucleobase. Mutation of the catalytic residue (e.g., D10 to A10) prevents cleavage of the edited (e.g., deaminated) strand containing the targeted residue (e.g., A or C). Such Cas9 variants can generate a single-strand DNA break (nick) at a specific location based on the gRNA-defined target sequence, leading to repair of the non-edited strand, ultimately resulting in a nucleobase change on the non-edited strand.

As non-limiting examples, a payload mRNA encodes a cytidine base editor (CBE), or an adenosine base editor (ABE), or variant thereof.

### Polynucleotide Programmable Nucleotide Binding Domain

In some embodiments, payload mRNA encodes a polynucleotide programmable nucleotide binding domain. Polynucleotide programmable nucleotide binding domains bind polynucleotides (e.g., RNA, DNA). A polynucleotide programmable nucleotide binding domain of a base editor can itself comprise one or more domains (e.g., one or more nuclease domains). In some embodiments, the nuclease domain of a polynucleotide programmable nucleotide binding domain can comprise an endonuclease or an exonuclease. An endonuclease can cleave a single strand of a double-stranded nucleic acid or both strands of a double-stranded nucleic acid molecule. In some embodiments, a nuclease domain of a polynucleotide programmable nucleotide binding domain can cut zero, one, or two strands of a target polynucleotide.

Fusion Proteins with Internal Insertions

In some embodiments, payload mRNA encodes a fusion protein. Fusion proteins comprise a heterologous polypeptide fused to a nucleic acid programmable nucleic acid binding protein, for example, a nucleic acid programmable DNA binding protein (napDNAbp). A heterologous polypeptide can be a polypeptide that is not found in the native or wild-type napDNAbp polypeptide sequence. The heterologous polypeptide can be fused to the napDNAbp at a C-terminal end of the napDNAbp, an N-terminal end of the napDNAbp, or inserted at an internal location of the napDNAbp.

In some embodiments, the heterologous polypeptide is inserted at an internal location of the napDNAbp. In some embodiments, the heterologous polypeptide is a deaminase (e.g., adenosine deaminase) or a functional fragment thereof. For example, a fusion protein can comprise a deaminase (e.g., adenosine deaminase) flanked by an N-terminal fragment and a C-terminal fragment of a Cas9 polypeptide. The deaminase in a fusion protein can be an adenosine deaminase. In some embodiments, the adenosine deaminase is a TadA (e.g., TadA*7.10 or a variant thereof).

In some embodiments, the fusion protein comprises the structure:
NH2-[N-terminal fragment of a napDNAbp]-[deaminase]-[C-terminal fragment of a napDNAbp]-COOH;
NH2-[N-terminal fragment of a Cas9]-[adenosine deaminase]-[C-terminal fragment of a Cas9]-COOH;
wherein each instance of “]-[” is an optional linker.

The deaminase can be a circular permutant deaminase. For example, the deaminase can be a circular permutant adenosine deaminase. In some embodiments, the deaminase is a circular permutant TadA, circularly permutated at amino acid residue 116 as numbered in the TadA reference sequence. In some embodiments, the deaminase is a circular permutant TadA, circularly permutated at amino acid residue 136 as numbered in the TadA reference sequence. In some embodiments, the deaminase is a circular permutant TadA, circularly permutated at amino acid residue 65 as numbered in the TadA reference sequence.

The fusion protein can comprise more than one deaminase. The fusion protein can comprise, for example, 1, 2, 3, 4, 5 or more deaminases. In some embodiments, the fusion protein comprises one deaminase. In some embodiments, the fusion protein comprises two deaminases. The two or more deaminases in a fusion protein can be an adenosine deaminase, cytidine deaminase, or a combination thereof. The two or more deaminases can be homodimers. The two or more deaminases can be heterodimers. The two or more deaminases can be inserted in tandem in the napDNAbp. In some embodiments, the two or more deaminases may not be in tandem in the napDNAbp.

In some embodiments, the napDNAbp in the fusion protein is a Cas9 polypeptide or a fragment thereof. The Cas9 polypeptide can be a variant Cas9 polypeptide. In some embodiments, the Cas9 polypeptide is a Cas9 nickase (nCas9) polypeptide or a fragment thereof. In some embodiments, the Cas9 polypeptide is a nuclease dead Cas9 (dCas9) polypeptide or a fragment thereof. The Cas9 polypeptide in a fusion protein can be a full-length Cas9 polypeptide. In some cases, the Cas9 polypeptide in a fusion protein may not be a full length Cas9 polypeptide. The Cas9 polypeptide can be truncated, for example, at a N-terminal or C-terminal end relative to a naturally-occurring Cas9 protein. The Cas9 polypeptide can be a circularly permuted Cas9 protein. The Cas9 polypeptide can be a fragment, a portion, or a domain of a Cas9 polypeptide that is still capable of binding the target polynucleotide and a guide nucleic acid sequence.

In some embodiments, the Cas9 polypeptide is a Streptococcus pyogenes Cas9 (SpCas9), Staphylococcus aureus Cas9 (SaCas9), Streptococcus thermophilus 1 Cas9 (St1Cas9), or fragments or variants thereof.

Fusion proteins comprising a heterologous catalytic domain flanked by N- and C-terminal fragments of a Cas9 polypeptide are also useful for base editing in the methods as described herein. Fusion proteins comprising Cas9 and one or more deaminase domains, e.g., adenosine deaminase, or comprising an adenosine deaminase domain flanked by Cas9 sequences are also useful for highly specific and efficient base editing of target sequences. In an embodiment, a chimeric Cas9 fusion protein contains a heterologous catalytic domain (e.g., adenosine deaminase, cytidine deaminase, or adenosine deaminase and cytidine deaminase) inserted within a Cas9 polypeptide. In some embodiments, the fusion protein comprises an adenosine deaminase domain and a cytidine deaminase domain inserted within a Cas9. In some embodiments, an adenosine deaminase is fused within a Cas9 and a cytidine deaminase is fused to the C-terminus. In some embodiments, an adenosine deaminase is fused within Cas9 and a cytidine deaminase fused to the N-terminus. In some embodiments, a cytidine deaminase is fused within Cas9 and an adenosine deaminase is fused to the C-terminus. In some embodiments, a cytidine deaminase is fused within Cas9 and an adenosine deaminase fused to the N-terminus.

Exemplary structures of a fusion protein with an adenosine deaminase and a cytidine deaminase and a Cas9 are provided as follows:
NH2-[Cas9(adenosine deaminase)]-[cytidine deaminase]-COOH;
NH2-[cytidine deaminase]-[Cas9(adenosine deaminase)]-COOH;
NH2-[Cas9(cytidine deaminase)]-[adenosine deaminase]-COOH; or
NH2-[adenosine deaminase]-[Cas9(cytidine deaminase)]-COOH.

In some embodiments, the “-” used in the general architecture above indicates the presence of an optional linker.

In various embodiments, the catalytic domain has DNA modifying activity (e.g., deaminase activity), such as adenosine deaminase activity. In some embodiments, the adenosine deaminase is a TadA (e.g., TadA*7.10). In some embodiments, the TadA is a TadA variant. In some embodiments, a TadA variant is fused within Cas9 and a cytidine deaminase is fused to the C-terminus. In some embodiments, a TadA variant is fused within Cas9 and a cytidine deaminase fused to the N-terminus. In some embodiments, a cytidine deaminase is fused within Cas9 and a TadA variant is fused to the C-terminus. In some embodiments, a cytidine deaminase is fused within Cas9 and a TadA variant fused to the N-terminus. Exemplary structures of a fusion protein with a TadA variant and a cytidine deaminase and a Cas9 are provided as follows:
NH2-[Cas9(TadA variant)]-[cytidine deaminase]-COOH;
NH2-[cytidine deaminase]-[Cas9(TadA variant)]-COOH;
NH2-[Cas9(cytidine deaminase)]-[TadA variant]-COOH; or
NH2-[TadA variant]-[Cas9(cytidine deaminase)]-COOH.

In some embodiments, the “-” used in the general architecture above indicates the presence of an optional linker.

In other embodiments, the fusion protein contains a nuclear localization signal (e.g., a bipartite nuclear localization signal).

In other embodiments, the Cas12b polypeptide contains a mutation that silences the catalytic activity of a RuvC domain. In other embodiments, the Cas12b polypeptide contains D574A, D829A and/or D952A mutations. In other embodiments, the fusion protein further contains a tag (e.g., an influenza hemagglutinin tag).

In some embodiments, the fusion protein comprises a napDNAbp domain (e.g., Cas12-derived domain) with an internally fused nucleobase editing domain (e.g., all or a portion of a deaminase domain, e.g., an adenosine deaminase domain). In some embodiments, the napDNAbp is a Cas12b.

By way of nonlimiting example, an adenosine deaminase (e.g., TadA*8.13) may be inserted into a BhCas12b to produce a fusion protein (e.g., TadA*8.13-BhCas12b) that effectively edits a nucleic acid sequence.

### Second RNA Molecule

In some embodiments, LNPs encapsulate a second RNA molecule. In some embodiments, a second RNA molecule facilitates the function of the first RNA molecule (i.e., the barcoded mRNA). In some embodiments, a second RNA molecule facilitates the function of a protein encoded by the first RNA molecule. In some embodiments, a second RNA molecule facilitates the function of a nuclease encoded by the first RNA molecule. In some embodiments, a second RNA molecule is a guide RNA (gRNA).

gRNA

In some embodiments, a second RNA molecule is a guide RNA (gRNA). In some embodiments, a second RNA molecule is a CRISPR RNA (crRNA). In some embodiments, a second RNA molecule is a trans-activating RNA (tracrRNA). In some embodiments, a second RNA molecule is a single guide RNA (sgRNA). In some embodiments, the sgRNA includes a loop between the tracrRNA and sgRNA. In some embodiments, the tracrRNA and crRNA form a hairpin loop. In some embodiments, sgRNA has at least two or more hairpins. In some embodiments, sgRNA has two, three, four or five hairpins.

In some embodiments, sgRNA includes a transcription termination sequence, which includes a polyT sequences comprising six nucleotides.

In some embodiments, the tracrRNA is a separate transcript, not contained with crRNA sequence in the same transcript.

When combined with a Cas protein, gRNA hybridizes with its target DNA, and guides Cas protein to cut, modify, or nick the target DNA.

In some embodiments, a second RNA molecule comprises a hairpin structure. In some embodiments, a second RNA molecule binds to an endogenous DNA or RNA.

In some embodiments, a second RNA molecule is about 10-500 nucleotides in length. In some embodiments, a second RNA molecule is about 15-150 nucleotides in length. In some embodiments, a second RNA molecule is about 50-100 nucleotides in length. In some embodiments, a second RNA molecule is about 10 nucleotides. In some embodiments, a second RNA molecule is about 15 nucleotides in length. In some embodiments, a second RNA molecule is about 17 nucleotides in length. In some embodiments, a second RNA molecule is about 20 nucleotides in length. In some embodiments, a second RNA molecule is about 25 nucleotides in length. In some embodiments, a second RNA molecule is about 30 nucleotides in length. In some embodiments, a second RNA molecule is about 35 nucleotides in length. In some embodiments, a second RNA molecule is about 40 nucleotides in length. In some embodiments, a second RNA molecule is about 45 nucleotides in length. In some embodiments, a second RNA molecule is about 50 nucleotides in length. In some embodiments, a second RNA molecule is about 60 nucleotides in length. In some embodiments, a second RNA molecule is about 70 nucleotides in length. In some embodiments, a second RNA molecule is about 80 nucleotides in length In some embodiments, a second RNA molecule is about 90 nucleotides in length. In some embodiments, a second RNA molecule is about 100 nucleotides in length. In some embodiments, a second RNA molecule is about 110 nucleotides in length. In some embodiments, a second RNA molecule is about 120 nucleotides in length. In some embodiments, a second RNA molecule is about 130 nucleotides in length. In some embodiments, a second RNA molecule is about 140 nucleotides in length. In some embodiments, a second RNA molecule is about 150 nucleotides in length. In some embodiments, a second RNA molecule is about 160 nucleotides in length. In some embodiments, a second RNA molecule is about 170 nucleotides in length. In some embodiments, a second RNA molecule is about 180 nucleotides in length. In some embodiments, a second RNA molecule is about 190 nucleotides in length. In some embodiments a second RNA molecule is about 200 nucleotides in length. In some embodiments, a second RNA molecule is about 220 nucleotides in length. In some embodiments, a second RNA molecule is about 250 nucleotides in length. In some embodiments, a second RNA molecule is about 300 nucleotides in length.

In some embodiments, a sgRNA is about 10-500 nucleotides in length. In some embodiments, a sgRNA is about 15-150 nucleotides in length. In some embodiments, a sgRNA is about 50-100 nucleotides in length. In some embodiments, a sgRNA is about 10 nucleotides in length. In some embodiments, a sgRNA is about 15 nucleotides in length. In some embodiments, a sgRNA is about 17 nucleotides in length. In some embodiments, a sgRNA is about 20 nucleotides in length. In some embodiments, a sgRNA is about 25 nucleotides in length. In some embodiments, a sgRNA is about 30 nucleotides in length. In some embodiments, a sgRNA is about 35 nucleotides in length. In some embodiments, a sgRNA is about 40 nucleotides in length. In some embodiments, a sgRNA is about 45 nucleotides in length. In some embodiments, a sgRNA is about 50 nucleotides in length. In some embodiments, a sgRNA is about 60 nucleotides in length. In some embodiments, a sgRNA is about 70 nucleotides in length. In some embodiments, a sgRNA is about 80 nucleotides in length. In some embodiments, a sgRNA is about 90 nucleotides in length. In some embodiments, a sgRNA is about 100 nucleotides in length. In some embodiments, a sgRNA is about 110 nucleotides in length. In some embodiments, a sgRNA is about 120 nucleotides in length. In some embodiments, a sgRNA is about 130 nucleotides in length. In some embodiments, a sgRNA is about 140 nucleotides in length. In some embodiments, a sgRNA is about 150 nucleotides in length. In some embodiments, a sgRNA is about 160 nucleotides in length. In some embodiments, a sgRNA is about 170 nucleotides in length. In some embodiments, a sgRNA is about 180 nucleotides in length. In some embodiments, a sgRNA is about 190 nucleotides in length. In some embodiments, a sgRNA is about 200 nucleotides in length. In some embodiments, a sgRNA is about 220 nucleotides in length. In some embodiments, a sgRNA is about 250 nucleotides in length. In some embodiments a sgRNA is about 300 nucleotides in length. In some embodiments, a sgRNA is about 400 nucleotides in length. In some embodiments, a sgRNA is about 500 nucleotides in length.

Payload mRNA: gRNA Ratios

In some embodiments, LNP comprises a payload mRNA and a second RNA molecule. In some embodiments, the payload mRNA and the second RNA is at a mass ratio from 1:10 to 10:1. In some embodiments, the payload mRNA and the second RNA is at a mass ratio from 1:5 to 5:1. In some embodiments, the payload mRNA and the second RNA is at a mass ratio from 1:4 to 4:1. In some embodiments, the b payload mRNA and the second RNA is at a mass ratio from 1:2 to 2:1. In some embodiments, the payload mRNA and the second RNA is at a mass ratio of 1:1. In some embodiments, the payload mRNA and the second RNA is at a mass ratio of 2:1. In some embodiments, the payload mRNA and the second RNA is at a mass ratio of 3:1. In some embodiments, the payload mRNA and the second RNA is at a mass ratio of 4:1. In some embodiments, the payload mRNA and the second RNA is at a mass ratio of 5:1. In some embodiments, the payload mRNA and the second RNA is at a mass ratio of 6:1. In some embodiments, the payload mRNA and the second RNA is at a mass ratio of 7:1. In some embodiments, the payload mRNA and the second RNA is at a mass ratio of 8:1. In some embodiments, the payload mRNA and the second RNA is at a mass ratio of 9:1. In some embodiments, the payload mRNA and the second RNA is at a mass ratio of 10:1. In some embodiments, the payload mRNA and the second RNA is at a mass ratio of 1:2. In some embodiments, the payload mRNA and the second RNA is at a mass ratio of 1:3. In some embodiments, the payload mRNA and the second RNA is at a mass ratio of 1:4. In some embodiments, the payload mRNA and the second RNA is at a mass ratio of 1:5. In some embodiments, the payload mRNA and the second RNA is at a mass ratio of 1:6. In some embodiments, the payload mRNA and the second RNA is at a mass ratio of 1:7. In some embodiments, the payload mRNA and the second RNA is at a mass ratio of 1:9. In some embodiments, the payload mRNA and the second RNA is at a mass ratio of 1:10.

### Nanoparticles

Methods described herein can be used to screen any nanoparticles. In particular, methods described herein can be used to screen any lipid nanoparticles.

Lipid nanoparticles include any one or more lipids. In some embodiments, the lipid nanoparticles (LNPs) may include one or more cationic/ionizable, PEGylated, structural, or other lipids, such as phospholipids. In some embodiments, a LNP comprises a cationic lipid, a non-cationic lipid, and a PEG-modified lipid. In some embodiments, a LNP comprises a cationic lipid, a non-cationic lipid, a PEG-modified lipid, and cholesterol. Cationic lipids include both permanently charged and ionizable lipids. The ionizable lipids, for example, comprise ionizable lipids including a central amine moiety and at least one biodegradable group. The lipids described herein may be advantageously used in lipid nanoparticles and lipid nanoparticle formulations for the delivery of therapeutic and/or prophylactics, such as a nucleic acid, to mammalian cells or organs.

Suitable LNPs can include typically known lipids in the art or any novel inventive lipids that are generated in the future. Exemplary lipids are described in the PCT patent application publications: WO 2015/095340, WO 2020/150320, WO 2020/219876, WO 2021/021634, WO 2021/113365, WO 2022/060871, WO 2017/075531, and WO 2021/141969, and the PCT Application No.: PCT/US2021/64339; the contents of each of which are incorporated by reference in their entirety.

Non-limiting examples are provided in the Table 1.

TABLE 1
Lipids Used for Gene Transfer

| Lipid | Abbreviation | Feature |
|---|---|---|
| 1,2-Dioleoyl-sn-glycero-3-phosphatidylcholine | DOPC | Helper |
| 1,2-Dioleoyl-sn-glycero-3-phosphatidylethanolamine | DOPE | Helper |
| Cholesterol | | Helper |
| N-[1-(2,3-Dioleyloxy)prophyl]N,N,N-trimethylammonium | DOTMA | Cationic |
| chloride | | |
| 1,2-Dioleoyloxy-3-trimethylammonium-propane | DOTAP | Cationic |
| Dioctadecylamidoglycylspermine | DOGS | Cationic |
| N-(3-Aminopropyl)-N,N-dimethyl-2,3-bis(dodecyloxy)-1- | GAP-DLRIE | Cationic |
| propanaminium bromide | | |
| Cetyltrimethylammonium bromide | CTAB | Cationic |
| 6-Lauroxyhexyl ornithinate | LHON | Cationic |
| 1-(2,3-Dioleoyloxypropyl)-2,4,6-trimethylpyridinium | 20c | Cationic |
| 2,3-Dioleyloxy-N-[2(sperminecarboxamido-ethyl]-N,N- | DOSPA | Cationic |
| dimethyl-1-propanaminium trifluoroacetate | | |
| 1,2-Dioleyl-3-trimethylammonium-propane | DOPA | Cationic |
| N-(2-Hydroxyethyl)-N,N-dimethyl-2,3-bis(tetradecyloxy)-1- | MDRIE | Cationic |
| propanaminium bromide | | |
| Dimyristooxypropyl dimethyl hydroxyethyl ammonium | DMRI | Cationic |
| bromide | | |
| 3β-[N-(N′,N′-Dimethylaminoethane)-carbamoyl]cholesterol | DC-Chol | Cationic |
| Bis-guanidium-tren-cholesterol | BGTC | Cationic |
| 1,3-Diodeoxy-2-(6-carboxy-spermyl)-propylamide | DOSPER | Cationic |
| Dimethyloctadecylammonium bromide | DDAB | Cationic |
| Dioctadecylamidoglicylspermidin | DSL | Cationic |
| rac-[(2,3-Dioctadecyloxypropyl)(2-hydroxyethyl)]- | CLIP-1 | Cationic |
| dimethylammonium chloride | | |
| rac-[2(2,3-Dihexadecyloxypropyl- | CLIP-6 | Cationic |
| oxymethyloxy)ethyl\|trimethylammoniun bromide | | |
| Ethyldimyristoylphosphatidylcholine | EDMPC | Cationic |
| 1,2-Distearyloxy-N,N-dimethyl-3-aminopropane | DSDMA | Cationic |
| 1,2-Dimyristoyl-trimethylammonium propane | DMTAP | Cationic |
| O,O′-Dimyristyl-N-lysyl aspartate | DMKE | Cationic |
| 1,2-Distearoyl-sn-glycero-3-ethylpho sphocholine | DSEPC | Cationic |
| N-Palmitoyl D-erythro-sphingosyl carbamoyl-spermine | CCS | Cationic |
| N-t-Butyl-N0-tetradecyl-3-tetradecylaminopropionamidine | diC14- | Cationic |
| | amidine | |
| Octadecenolyoxy[ethyl-2-heptadecenyl-3 hydroxyethyl] | DOTIM | Cationic |
| imidazolinium chloride | | |
| N1-Cholesteryloxycarbonyl-3,7-diazanonane-1,9-diamine | CDAN | Cationic |
| 2-(3-[Bis(3-amino-propyl)-amino]propylamino)-N- | RPR209120 | Cationic |
| ditetradecylcarbamoylme-ethyl-acetamide | | |
| 1,2-dilinoleyloxy-3-dimethylaminopropane | DLinDMA | Cationic |
| 2,2-dilinoleyl-4-dimethylaminoethyl-[1,3]-dioxolane | DLin-KC2- | Cationic |
| | DMA | |
| dilinoleyl-methyl-4-dimethylaminobutyrate | DLin-MC3- | Cationic |
| | DMA | |

Table 2 lists exemplary polymers for use in gene transfer and/or nanoparticle formulations.

TABLE 2
Polymers Used for Gene Transfer

| Polymer | Abbreviation |
|---|---|
| Poly(ethylene)glycol | PEG |
| Polyethylenimine | PEI |
| Dithiobis (succinimidylpropionate) | DSP |
| Dimethyl-3,3′-dithiobispropionimidate | DTBP |
| Poly(ethylene imine)biscarbamate | PEIC |
| Poly(L-lysine) | PLL |
| Histidine modified PLL | |
| Poly(N-vinylpyrrolidone) | PVP |
| Poly(propylenimine) | PPI |
| Poly(amidoamine) | PAMAM |
| Poly(amidoethylenimine) | SS-PAEI |
| Triethylenetetramine | TETA |
| Poly(β-aminoester) | |
| Poly(4-hydroxy-L-proline ester) | PHP |
| Poly(allylamine) | |
| Poly(α-[4-aminobutyl]-L-glycolic acid) | PAGA |
| Poly(D,L-lactic-co-glycolic acid) | PLGA |
| Poly(N-ethyl-4-vinylpyridinium bromide) | |
| Poly(phosphazene)s | PPZ |
| Poly(phosphoester)s | PPE |
| Poly(phosphoramidate)s | PPA |
| Poly(N-2-hydroxypropylmethacrylamide) | pHPMA |
| Poly (2-(dimethylamino)ethyl methacrylate) | pDMAEMA |
| Poly(2-aminoethyl propylene phosphate) | PPE-EA |
| Chitosan | |
| Galactosylated chitosan | |
| N-Dodacylated chitosan | |
| Histone | |
| Collagen | |
| Dextran-spermine | D-SPM |

### Non-Cationic Lipids

In some embodiments, LNP comprises one or more non-cationic (“helper”) lipids. As used herein, the phrase “non-cationic lipid” refers to any neutral, zwitterionic or anionic lipid. As used herein, the phrase “anionic lipid” refers to any of a number of lipid species that carry a net negative charge at a selected H, such as physiological pH. Non-cationic lipids include, but are not limited to, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoylphosphatidylethanolamine (DOPE), palmitoyloleoylphosphatidylcholine (POPC), palmitoyloleoyl-phosphatidylethanolamine (POPE), dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidyl-ethanolamine (DSPE), 16-O-monomethyl PE, 16-O-dimethyl PE, 18-1-trans PE, 1-stearoyl-2-oleoyl-phosphatidyethanolamine (SOPE), or a mixture thereof.

In some embodiments, such non-cationic lipids may be used alone, but are preferably used in combination with other excipients, for example, cationic lipids. In some embodiments, the non-cationic lipid may comprise a molar ratio of about 5% to about 90%, or about 10% to about 70% of the total lipid present in a liposome. In some embodiments, a non-cationic lipid is a neutral lipid, i.e., a lipid that does not carry a net charge in the conditions under which the composition is formulated and/or administered. In some embodiments, the percentage of non-cationic lipid in a liposome may be greater than 5%, greater than 10%, greater than 20%, greater than 30%, or greater than 40%.

### PEG-Modified Lipids

As used herein, the term “PEG lipid” refers to polyethylene glycol (PEG)-modified lipids. PEG lipid and PEG-modified lipid are used interchangeably. PEG lipids include PEG-modified phosphatidylethanolamine and phosphatidic acid, PEG-ceramide conjugates (e.g., PEG-CerC14 or PEG-CerC20), PEG-modified dialkylamines and PEG-modified 1,2-diacyloxypropan-3-amines. Such lipids are also referred to as PEGylated lipids. In some embodiments, a PEG lipid can be PEG-c-DOMG, PEG-DMG, PEG-DLPE, PEG-DMPE, PEG-DPPC, or a PEG-DSPE lipid.

In some embodiments, the PEG lipid includes, but are not limited to, 1,2-dimyristoyl-sn-glycerol methoxypoly ethylene glycol (PEG-DMG), 1,2-distearoyl-sn-glycero-3-phosphoethanolamine-N-[amino(polyethylene glycol)](PEG-DSPE), PEG-disteryl glycerol (PEG-DSG), PEG-dipalmetoleyl, PEG-dioleyl, PEG-distearyl, PEG-diacylglycamide (PEG-DAG), PEG-dipalmitoyl phosphatidylethanolamine (PEG-DPPE), or PEG-1, 2-dimyristyloxlpropy 1-3-amine (PEG-c-DMA).

In some embodiments, the PEG lipid is selected from the group consisting of a PEG-modified phosphatidylethanolamine, a PEG-modified phosphatidic acid, a PEG-modified ceramide, a PEG-modified dialkylamine, a PEG-modified diacylglycerol, a PEG-modified dialkylglycerol, and mixtures thereof.

In some embodiments, the lipid moiety of the PEG lipids includes those having lengths of from about Ci4to about C22, e.g., from about Ci4 to about Ci6. In some embodiments, a PEG moiety, for example an mPEG-NEb, has a size of about 1000, 2000, 5000, 10,000, 15,000 or 20,000 daltons. In some embodiments, the PEG lipid is PEG2k-DMG.

In some embodiments, the lipid nanoparticles described herein can comprise a PEG lipid which is a non-diffusible PEG. Non-limiting examples of non-diffusible PEGs include PEG-DSG and PEG-DSPE. [0363] PEG lipids are known in the art, such as those described in U.S. Pat. No. 8,158,601 and International Publ. No. WO 2015/130584 A2, which are incorporated herein by reference in their entirety.

In general, some of the other lipid components (e.g., PEG lipids) of various formulae, described herein may be synthesized as described International Patent Application No. PCT/US2016/000129, filed Dec. 10, 2016, entitled “Compositions and Methods for Delivery of Therapeutic Agents,” which is incorporated by reference in its entirety.

The lipid component of a lipid nanoparticle or lipid nanoparticle formulation may include one or more molecules comprising polyethylene glycol, such as PEG or PEG-modified lipids. Such species may be alternately referred to as PEGylated lipids. A PEG lipid is a lipid modified with polyethylene glycol. A PEG lipid may be selected from the non-limiting group including PEG-modified phosphatidylethanolamines, PEG-modified phosphatidic acids, PEG-modified ceramides, PEG-modified dialkylamines, PEG-modified diacylglycerols, PEG-modified dialkylglycerols, and mixtures thereof. In some embodiments, a PEG lipid may be PEG-c-DOMG, PEG-DMG, PEG-DLPE, PEG-DMPE, PEG-DPPC, or a PEG-DSPE lipid.

### Cholesterols

In some embodiments, LNP comprises one or more cholesterol-based lipids. For example, suitable cholesterol-based lipids include, for example, DC-Choi (N,N-dimethyl-N-ethylcarboxamidocholesterol), 1,4-bis(3-N-oleylamino-propyl)piperazine (Gao, et al. Biochem. Biophys. Res. Comm. 179, 280 (1991); Wolf et al. BioTechniques 23, 139 (1997); U.S. Pat. No. 5,744,335), or ICE.

### Compositions

Provided here also include LNPs that are characterized according to the in vivo screening methods described herein. The LNP can be used to deliver a payload to the tissue and/or cell of interest.

Compositions comprising a LNP identified through the present methods and a payload mRNA described herein are provided. The LNP composition can preferentially deliver a payload (e.g., barcoded mRNA) to a target tissue or cell which is identified by practicing the present methods. The composition may further comprise a second molecule encapsulated in the same LNP. In some examples, the second molecule is another RNA (e.g., a gRNA, siRNA, and microRNA etc.). In some embodiments, the second RNA of the composition is a gRNA.

### EXAMPLES

The following examples describe some of the preferred modes of making and practicing the present invention. However, it should be understood that these examples are for illustrative purposes only and are not meant to limit the scope of the invention.

### Example 1: Design of Barcodes and Production of Barcoded mRNA

Hundreds of different RNA barcodes 8 nucleotides or 20 nucleotides in length were designed. Each unique barcode sequence was designed to allow discernment of each lipid nanoparticle, to not lead to changes in the stability of an mRNA to be barcoded, and to not be homologous to any miRNA binding sites and therefore minimize or avoid triggering RISC-mediated premature degradation. The barcode sequences were also designed to minimize any secondary structure (e.g., low ΔG).

Some exemplary barcode sequences are listed in Tables 3 and 4.

TABLE 3
Exemplary 8 nucleotides barcode sequences

| ID | Barcode | Reverse complement (with barcode sequence) |
|---|---|---|
| UTR_ | AACATCGC | TTTTTTTTTTTTTTTTTTTTTTTTTTTTTTTTTTTTTTTTTTTTTTT |
| BC_9 | (SEQ ID NO. | TTTTTTTTTTTTTTTTTTTTTTTTTTTTTTTTTTTTTTTTTTTTTTT |
| | 3) | TTTTTTTTTTTTTTTTTTTTTTTTTTCTTCCTACTCAGGCTTTATTC |
| | | AAAGACGCGATGTTCAAGAGGTACAGGTGC (SEQ ID NO. 4) |
| UTR_ | TCAACACC | TTTTTTTTTTTTTTTTTTTTTTTTTTTTTTTTTTTTTTTTTTTTTTT |
| BC_10 | (SEQ ID NO. | TTTTTTTTTTTTTTTTTTTTTTTTTTTTTTTTTTTTTTTTTTTTTTT |
| | 5) | TTTTTTTTTTTTTTTTTTTTTTTTTTCTTCCTACTCAGGCTTTATTC |
| | | AAAGACGGTGTTGACAAGAGGTACAGGTGC (SEQ ID NO. 6) |
| UTR_ | TTCGATCC | TTTTTTTTTTTTTTTTTTTTTTTTTTTTTTTTTTTTTTTTTTTTTTT |
| BC_11 | (SEQ ID NO. | TTTTTTTTTTTTTTTTTTTTTTTTTTTTTTTTTTTTTTTTTTTTTTT |
| | 7) | TTTTTTTTTTTTTTTTTTTTTTTTTTCTTCCTACTCAGGCTTTATTC |
| | | AAAGACGGATCGAACAAGAGGTACAGGTGC (SEQ ID NO. 8) |
| UTR_ | TCTCATCC | TTTTTTTTTTTTTTTTTTTTTTTTTTTTTTTTTTTTTTTTTTTTTTT |
| BC_12 | (SEQ ID NO. | TTTTTTTTTTTTTTTTTTTTTTTTTTTTTTTTTTTTTTTTTTTTTTT |
| | 9) | TTTTTTTTTTTTTTTTTTTTTTTTTTCTTCCTACTCAGGCTTTATTC |
| | | AAAGACGGATGAGACAAGAGGTACAGGTGC (SEQ ID NO. 10) |
| UTR_ | ATCACCTC | TTTTTTTTTTTTTTTTTTTTTTTTTTTTTTTTTTTTTTTTTTTTTTT |
| BC_13 | (SEQ ID NO. | TTTTTTTTTTTTTTTTTTTTTTTTTTTTTTTTTTTTTTTTTTTTTTT |
| | 11) | TTTTTTTTTTTTTTTTTTTTTTTTTTCTTCCTACTCAGGCTTTATTC |
| | | AAAGACGAGGTGATCAAGAGGTACAGGTGC (SEQ ID NO. 12) |
| UTR_ | AATCCGCT | TTTTTTTTTTTTTTTTTTTTTTTTTTTTTTTTTTTTTTTTTTTTTTT |
| BC_14 | (SEQ ID NO. | TTTTTTTTTTTTTTTTTTTTTTTTTTTTTTTTTTTTTTTTTTTTTTT |
| | 13) | TTTTTTTTTTTTTTTTTTTTTTTTTTCTTCCTACTCAGGCTTTATTC |
| | | AAAGACAGCGGATTCAAGAGGTACAGGTGC (SEQ ID NO. 14) |
| UTR_ | ATGTCGCT | TTTTTTTTTTTTTTTTTTTTTTTTTTTTTTTTTTTTTTTTTTTTTTT |
| BC_15 | (SEQ ID NO. | TTTTTTTTTTTTTTTTTTTTTTTTTTTTTTTTTTTTTTTTTTTTTTT |
| | 15) | TTTTTTTTTTTTTTTTTTTTTTTTTTCTTCCTACTCAGGCTTTATTC |
| | | AAAGACAGCGACATCAAGAGGTACAGGTGC (SEQ ID NO. 16) |
| UTR_ | TCTTCGCT | TTTTTTTTTTTTTTTTTTTTTTTTTTTTTTTTTTTTTTTTTTTTTTT |
| BC_16 | (SEQ ID NO. | TTTTTTTTTTTTTTTTTTTTTTTTTTTTTTTTTTTTTTTTTTTTTTT |
| | 17) | TTTTTTTTTTTTTTTTTTTTTTTTTTCTTCCTACTCAGGCTTTATTC |
| | | AAAGACAGCGAAGACAAGAGGTACAGGTGC (SEQ ID NO. 18) |

TABLE 4
Exemplary 20 nucleotides barcode sequences

| ID | Barcode | Reverse complement |
|---|---|---|
| UTR_ | TCGTAGAT | TTTTTTTTTTTTTTTTTTTTTTTTTTTTTTTTTTTTTTTTTTTTTTT |
| 20BC_ | GTCCGCAG | TTTTTTTTTTTTTTTTTTTTTTTTTTTTTTTTTTTTTTTTTTTTTTT |
| 1 | TCTC (SEQ | TTTTTTTTTTTTTTTTTTTTTTTTTTCTTCCTACTCAGGCTTTATTC |
| | ID NO. 19) | AAAGACGAGACTGCGGACATCTACGACAAGAGGTACAGGTGC |
| | | (SEQ ID NO. 20) |
| UTR_ | TCTCGCTT | TTTTTTTTTTTTTTTTTTTTTTTTTTTTTTTTTTTTTTTTTTTTTTT |
| 20BC_ | ACCTCACT | TTTTTTTTTTTTTTTTTTTTTTTTTTTTTTTTTTTTTTTTTTTTTTT |
| 2 | CGAA (SEQ | TTTTTTTTTTTTTTTTTTTTTTTTTTCTTCCTACTCAGGCTTTATTC |
| | ID NO. 21) | AAAGACTTCGAGTGAGGTAAGCGAGACAAGAGGTACAGGTGC |
| | | (SEQ ID NO. 22) |
| UTR_ | TTCGTAGA | TTTTTTTTTTTTTTTTTTTTTTTTTTTTTTTTTTTTTTTTTTTTTTT |
| 20BC_ | TGTCGGTC | TTTTTTTTTTTTTTTTTTTTTTTTTTTTTTTTTTTTTTTTTTTTTTT |
| 3 | CGGA (SEQ | TTTTTTTTTTTTTTTTTTTTTTTTTTCTTCCTACTCAGGCTTTATTC |
| | ID NO. 23) | AAAGACTCCGGACCGACATCTACGAACAAGAGGTACAGGTGC |
| | | (SEQ ID NO. 24) |
| UTR_ | TTCGAATC | TTTTTTTTTTTTTTTTTTTTTTTTTTTTTTTTTTTTTTTTTTTTTTT |
| 20BC_ | CTATCACG | TTTTTTTTTTTTTTTTTTTTTTTTTTTTTTTTTTTTTTTTTTTTTTT |
| 4 | CTCC (SEQ | TTTTTTTTTTTTTTTTTTTTTTTTTTCTTCCTACTCAGGCTTTATTC |
| | ID NO. 25) | AAAGACGGAGCGTGATAGGATTCGAACAAGAGGTACAGGTGC |
| | | (SEQ ID NO. 26) |
| UTR_ | ATCCTTCC | TTTTTTTTTTTTTTTTTTTTTTTTTTTTTTTTTTTTTTTTTTTTTTT |
| 20BC_ | TAACCGAG | TTTTTTTTTTTTTTTTTTTTTTTTTTTTTTTTTTTTTTTTTTTTTTT |
| 5 | CATT (SEQ | TTTTTTTTTTTTTTTTTTTTTTTTTTCTTCCTACTCAGGCTTTATTC |
| | ID NO. 27) | AAAGACAATGCTCGGTTAGGAAGGATCAAGAGGTACAGGTGC |
| | | (SEQ ID NO. 28) |
| UTR_ | CGCGTCCT | TTTTTTTTTTTTTTTTTTTTTTTTTTTTTTTTTTTTTTTTTTTTTTT |
| 20BC_ | ATCTTATG | TTTTTTTTTTTTTTTTTTTTTTTTTTTTTTTTTTTTTTTTTTTTTTT |
| 6 | TTCC (SEQ | TTTTTTTTTTTTTTTTTTTTTTTTTTCTTCCTACTCAGGCTTTATTC |
| | ID NO. 29) | AAAGACGGAACATAAGATAGGACGCGCAAGAGGTACAGGTGC |
| | | (SEQ ID NO. 30) |
| UTR_ | ATATCCGC | TTTTTTTTTTTTTTTTTTTTTTTTTTTTTTTTTTTTTTTTTTTTTTT |
| 20BC_ | AGTCAACG | TTTTTTTTTTTTTTTTTTTTTTTTTTTTTTTTTTTTTTTTTTTTTTT |
| 7 | TTAG (SEQ | TTTTTTTTTTTTTTTTTTTTTTTTTTCTTCCTACTCAGGCTTTATTC |
| | ID NO. 31) | AAAGACCTAACGTTGACTGCGGATATCAAGAGGTACAGGTGC |
| | | (SEQ ID NO. 32) |
| UTR_ | GCTGAGTC | TTTTTTTTTTTTTTTTTTTTTTTTTTTTTTTTTTTTTTTTTTTTTTT |
| 20BC_ | GGTTGTCT | TTTTTTTTTTTTTTTTTTTTTTTTTTTTTTTTTTTTTTTTTTTTTTT |
| 8 | GTAA (SEQ | TTTTTTTTTTTTTTTTTTTTTTTTTTCTTCCTACTCAGGCTTTATTC |
| | ID NO. 33) | AAAGACTTACAGACAACCGACTCAGCCAAGAGGTACAGGTGC |
| | | (SEQ ID NO. 34) |
| UTR_ | CCATTCTA | TTTTTTTTTTTTTTTTTTTTTTTTTTTTTTTTTTTTTTTTTTTTTTT |
| 20BC_ | TTATGCTA | TTTTTTTTTTTTTTTTTTTTTTTTTTTTTTTTTTTTTTTTTTTTTTT |
| 9 | CCGT (SEQ | TTTTTTTTTTTTTTTTTTTTTTTTTTCTTCCTACTCAGGCTTTATTC |
| | ID NO. 35) | AAAGACACGGTAGCATAATAGAATGGCAAGAGGTACAGGTGC |
| | | (SEQ ID NO. 36) |
| UTR_ | AACACGGC | TTTTTTTTTTTTTTTTTTTTTTTTTTTTTTTTTTTTTTTTTTTTTTT |
| 20BC_ | CAGTTACT | TTTTTTTTTTTTTTTTTTTTTTTTTTTTTTTTTTTTTTTTTTTTTTT |
| 10 | AACA (SEQ | TTTTTTTTTTTTTTTTTTTTTTTTTTCTTCCTACTCAGGCTTTATTC |
| | ID NO. 37) | AAAGACTGTTAGTAACTGGCCGTGTTCAAGAGGTACAGGTGC |
| | | (SEQ ID NO. 38) |
| UTR_ | GCGTCTGA | TTTTTTTTTTTTTTTTTTTTTTTTTTTTTTTTTTTTTTTTTTTTTTT |
| 20BC_ | AGAACCTG | TTTTTTTTTTTTTTTTTTTTTTTTTTTTTTTTTTTTTTTTTTTTTTT |
| 11 | TATA (SEQ | TTTTTTTTTTTTTTTTTTTTTTTTTTCTTCCTACTCAGGCTTTATTC |
| | ID NO. 39) | AAAGACTATACAGGTTCTTCAGACGCCAAGAGGTACAGGTGC |
| | | (SEQ ID NO. 40) |
| UTR_ | TATGCCAT | TTTTTTTTTTTTTTTTTTTTTTTTTTTTTTTTTTTTTTTTTTTTTTT |
| 20BC_ | CCTCGTCC | TTTTTTTTTTTTTTTTTTTTTTTTTTTTTTTTTTTTTTTTTTTTTTT |
| 12 | TATT (SEQ | TTTTTTTTTTTTTTTTTTTTTTTTTTCTTCCTACTCAGGCTTTATTC |
| | ID NO. 41) | AAAGACAATAGGACGAGGATGGCATACAAGAGGTACAGGTGC |
| | | (SEQ ID NO. 42) |
| UTR_ | TCAATCTC | TTTTTTTTTTTTTTTTTTTTTTTTTTTTTTTTTTTTTTTTTTTTTTT |
| 20BC_ | ACTGGCTG | TTTTTTTTTTTTTTTTTTTTTTTTTTTTTTTTTTTTTTTTTTTTTTT |
| 13 | GAAC (SEQ | TTTTTTTTTTTTTTTTTTTTTTTTTTCTTCCTACTCAGGCTTTATTC |
| | ID NO. 43) | AAAGACGTTCCAGCCAGTGAGATTGACAAGAGGTACAGGTGC |
| | | (SEQ ID NO. 44) |
| UTR_ | TTCCAGTC | TTTTTTTTTTTTTTTTTTTTTTTTTTTTTTTTTTTTTTTTTTTTTTT |
| 20BC_ | CGCTACGT | TTTTTTTTTTTTTTTTTTTTTTTTTTTTTTTTTTTTTTTTTTTTTTT |
| 14 | AACA (SEQ | TTTTTTTTTTTTTTTTTTTTTTTTTTCTTCCTACTCAGGCTTTATTC |
| | ID NO. 45) | AAAGACTGTTACGTAGCGGACTGGAACAAGAGGTACAGGTGC |
| | | (SEQ ID NO. 46) |
| UTR_ | CCTTAGTC | TTTTTTTTTTTTTTTTTTTTTTTTTTTTTTTTTTTTTTTTTTTTTTT |
| 20BC_ | TCACCGAG | TTTTTTTTTTTTTTTTTTTTTTTTTTTTTTTTTTTTTTTTTTTTTTT |
| 15 | CCGA (SEQ | TTTTTTTTTTTTTTTTTTTTTTTTTTCTTCCTACTCAGGCTTTATTC |
| | ID NO. 47) | AAAGACTCGGCTCGGTGAGACTAAGGCAAGAGGTACAGGTGC |
| | | (SEQ ID NO. 48) |
| UTR_ | GCGTTCAA | TTTTTTTTTTTTTTTTTTTTTTTTTTTTTTTTTTTTTTTTTTTTTTT |
| 20BC_ | TGTTCACC | TTTTTTTTTTTTTTTTTTTTTTTTTTTTTTTTTTTTTTTTTTTTTTT |
| 16 | TGTC (SEQ | TTTTTTTTTTTTTTTTTTTTTTTTTTCTTCCTACTCAGGCTTTATTC |
| | ID NO. 49) | AAAGACGACAGGTGAACATTGAACGCCAAGAGGTACAGGTGC |
| | | (SEQ ID NO. 50) |

A standard 3′UTR sequence from the mouse alpha globin 3′UTR was used to for the barcode insertion. Table 5 shows the 3-UTR sequence and the position of the barcode sequence.

3′UTR sequence from mouse alpha-globin:

(SEQ ID NO. 51)
5′TTAATTAAGCTGCCTTCTGCGGGGCTTGCCTTCTGGCCATGCCCTTCT
TCTCTCCCTTGCACCTGTACCTCTTGGTCTTTGAATAAAGCCTGAGTAGG
AAG3′.

TABLE 5
3′-UTR and barcode position

| | Sequence (5′-3′) |
|---|---|
| Sequence before | TTAATTAAGCTGCCTTCTGCGGGGCTTGCC |
| first binding | TTCTGGCCATGCCCTTCTTCTCTCCCTTGC |
| site | ACCTGTACCTCTTG (SEQ ID NO. 52) |
| Barcode (8nt) | (8nt barcode) |
| Sequence after | GTCTTTGAATAAAGCCTGAGTAGGAAG |
| the barcode | (SEQ ID NO. 53) |
| Full length of | TTAATTAAGCTGCCTTCTGCGGGGCTTGCC |
| 3′-UTR (barcoded) | TTCTGGCCATGCCCTTCTTCTCTCCCTTGC |
| | ACCTGTACCTCTTGNNNNNNNN(8nt |
| | Barcode)GTCTTTGAATAAAGCCTGAGTA |
| | GGAAG (SEQ ID NO. 1) |

The primers for the production of barcoded mRNAs from a PCR template are shown in Table 6.

TABLE 6
Primers for producing IVT template

| Name | Reverse complement (with barcode sequence) |
|---|---|
| UTR_BC_1 | TTTTTTTTTTTTTTTTTTTTTTTTTTTTTTTTTTTTTTTTTTTTTTTTTTT |
| | TTTTTTTTTTTTTTTTTTTTTTTTTTTTTTTTTTTTTTTTTTTTTTTTTTT |
| | TTTTTTTTTTTTTTTTTTCTTCCTACTCAGGCTTTATTCAAAGACTTCGCG |
| | ATCAAGAGGTACAGGTGC (SEQ ID NO. 54) |
| UTR_BC_2 | TTTTTTTTTTTTTTTTTTTTTTTTTTTTTTTTTTTTTTTTTTTTTTTTTTT |
| | TTTTTTTTTTTTTTTTTTTTTTTTTTTTTTTTTTTTTTTTTTTTTTTTTTT |
| | TTTTTTTTTTTTTTTTTTCTTCCTACTCAGGCTTTATTCAAAGACTCGGTA |
| | GACAAGAGGTACAGGTGC (SEQ ID NO. 55) |
| UTR_BC_3 | TTTTTTTTTTTTTTTTTTTTTTTTTTTTTTTTTTTTTTTTTTTTTTTTTTT |
| | TTTTTTTTTTTTTTTTTTTTTTTTTTTTTTTTTTTTTTTTTTTTTTTTTTT |
| | TTTTTTTTTTTTTTTTTTCTTCCTACTCAGGCTTTATTCAAAGACTCGACG |
| | AACAAGAGGTACAGGTGC (SEQ ID NO. 56) |
| UTR_BC_4 | TTTTTTTTTTTTTTTTTTTTTTTTTTTTTTTTTTTTTTTTTTTTTTTTTTT |
| | TTTTTTTTTTTTTTTTTTTTTTTTTTTTTTTTTTTTTTTTTTTTTTTTTTT |
| | TTTTTTTTTTTTTTTTTTCTTCCTACTCAGGCTTTATTCAAAGACTCGAAC |
| | GACAAGAGGTACAGGTGC (SEQ ID NO. 57) |
| UTR_BC_5 | TTTTTTTTTTTTTTTTTTTTTTTTTTTTTTTTTTTTTTTTTTTTTTTTTTT |
| | TTTTTTTTTTTTTTTTTTTTTTTTTTTTTTTTTTTTTTTTTTTTTTTTTTT |
| | TTTTTTTTTTTTTTTTTTCTTCCTACTCAGGCTTTATTCAAAGACCGGATA |
| | GACAAGAGGTACAGGTGC (SEQ ID NO. 58) |
| UTR_BC_6 | TTTTTTTTTTTTTTTTTTTTTTTTTTTTTTTTTTTTTTTTTTTTTTTTTTT |
| | TTTTTTTTTTTTTTTTTTTTTTTTTTTTTTTTTTTTTTTTTTTTTTTTTTT |
| | TTTTTTTTTTTTTTTTTTCTTCCTACTCAGGCTTTATTCAAAGACGTTCGG |
| | TTCAAGAGGTACAGGTGC (SEQ ID NO. 59) |
| UTR_BC_7 | TTTTTTTTTTTTTTTTTTTTTTTTTTTTTTTTTTTTTTTTTTTTTTTTTTT |
| | TTTTTTTTTTTTTTTTTTTTTTTTTTTTTTTTTTTTTTTTTTTTTTTTTTT |
| | TTTTTTTTTTTTTTTTTTCTTCCTACTCAGGCTTTATTCAAAGACGTCGGT |
| | AACAAGAGGTACAGGTGC (SEQ ID NO. 60) |
| UTR_BC_8 | TTTTTTTTTTTTTTTTTTTTTTTTTTTTTTTTTTTTTTTTTTTTTTTTTTT |
| | TTTTTTTTTTTTTTTTTTTTTTTTTTTTTTTTTTTTTTTTTTTTTTTTTTT |
| | TTTTTTTTTTTTTTTTTTCTTCCTACTCAGGCTTTATTCAAAGACGCCGAT |
| | ATCAAGAGGTACAGGTGC (SEQ ID NO. 61) |

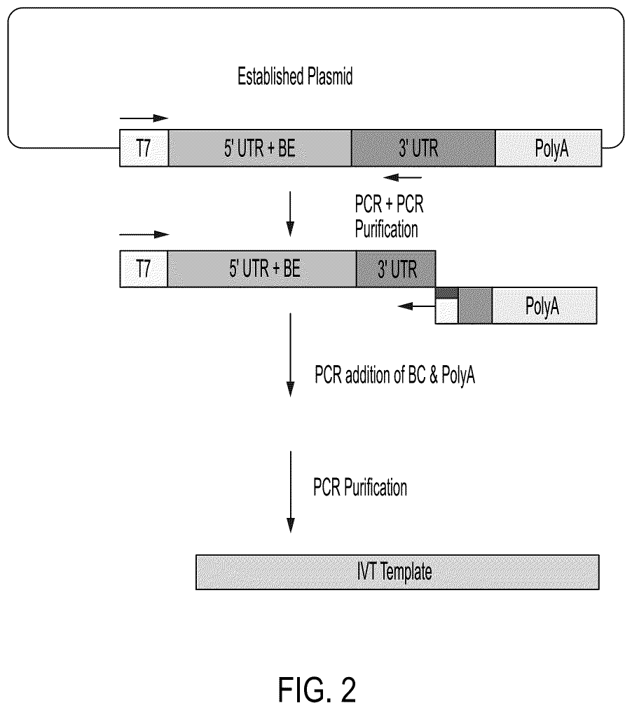

As shown in FIG. 2, a plasmid comprising a coding sequence of a protein was generated. The protein coding sequence was extracted and amplified from the plasmid by PCR using primers targeting the T7 promoter and the 3′UTR. The extracted sequence was further amplified using the primers specific the T7 promoter and the primers comprising barcodes in Table 6. The PCR amplified and generated an in vitro transcription (JVT) template for producing a barcoded mRNA. The barcoded mRNA from the PCR template will comprise the sequence of:

(SEQ ID NO. 1)
5′TTAATTAAGCTGCCTTCTGCGGGGCTTGCCTTCTGGCCATGCCCTTCT
TCTCTCCCTTGCACCTGTACCTCTTG(NNNNNNNN)GTCTTTGAATAAAG
CCTGAGTAGGAAG-3′, wherein each of N is A, T, G or
C.

The barcoded mRNAs produced from the PCR templates using the primers in Table 6 were purified and participated using LiCl. Quality test indicated that the eight barcoded mRNAs produced from the PCR templates all have a high quality. See Table 7 below.

TABLE 7
Quality of mRNA amplified using PCT IVT template

| | | | | Residual | Residual DNA | IFN Beta |
|---|---|---|---|---|---|---|
| Sample | | | Endotoxin | Protein | (ng DNA/mg | Induction |
| ID | % Capped | % FLP | (EU/mL) | (mg/mL) | RNA) | (fg/mL) |
| 1451 | 99.51 | 81.7 | <0.5 | 0.0079 | 0.6814 | 2829 |
| 1452 | 99.54 | 80.8 | <0.5 | 0.0084 | 0.3487 | 2238 |
| 1453 | 99.55 | 80.8 | <0.5 | 0.0084 | 4.6186 | 1796 |
| 1454 | 99.35 | 80.8 | <0.5 | 0.0089 | 4.2094 | 1254 |
| 1455 | 99.54 | 76.8 | 0.551 | 0.0091 | 5.7724 | 1236 |
| 1456 | 99.41 | 81.3 | <0.524 | 0.0093 | 2.8904 | 3114 |
| 1457 | 99.28 | 80.5 | <0.5 | 0.009 | 8.2807 | 2704 |
| 1458 | 99.71 | 81.6 | <0.5 | 0.0082 | 6.1021 | 1137 |
| Averagex | 99.48625 | 80.5375 | 0.551 | 0.00865 | 4.1129625 | 2038.5 |

### Example 2. Validation of Barcoded Payload mRNAs

Validation of Barcoded Payload mRNA Expression

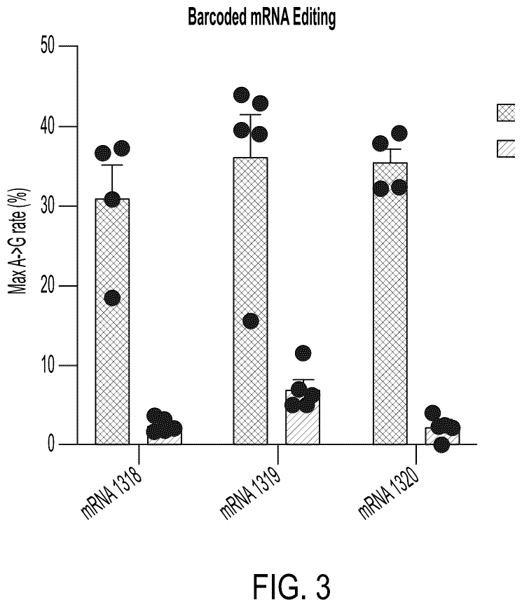

To test if the barcoded mRNA encoding a protein can express the encoded base editor, three barcoded mRNAs with different barcode sequences (mRNA #1318, mRNA #1319 and mRNA #1320) were formulated with lipid nanoparticle; each LNP is formulated with one barcoded mRNA encoding the base editor and a sgRNA (sg23). Three mRNA and sgRNA-loaded LNPs were administered (IV injection) to mice at a dose of 0.05 mg/kg or 0.25 mg/kg (n=5 for each group). Saline was injected to a group of mice as negative control (n=5). Four days after the administration, the mice from each group were sacrificed and base editing was tested. The results indicate that the three LNPs, each carrying differently barcoded payload mRNAs, have similar base editing efficiency (FIG. 3). The observation suggests the presence of different 8nt barcodes in the 3′ UTR do not influence editing efficiency of the enzyme.

Measurement of Barcoded Payload mRNA Distribution-FAI

Each barcode relative abundance is first determined by sequencing the barcode counts in an administration LNP pool (relative abundance in “input”). After administration, one or more tissues and cells are isolated and total mRNA are extracted from the tissues and cells. The barcodes in the mRNA extracts are sequenced for their relative abundance in the RNA extracts. The FAI value of a RNA barcode is calculated by normalizing the relative abundance in the RNA barcode sequence counts in the isolated samples to its relative abundance in the administration input. The value ‘1’ represents a LNP appearing at the same frequency in the isolated sample as it does in the administration pool, representing that it displays neutral tropism to the cell-type measured relative to other LNP populations in that same administration pool. The FAI then indicates the performance of an LNP relative to the input LNP composition. FIG. 4 shows the flowchart for calculating FAI. The following formula is used for calculating FAI:

$$FAI = \frac{R e l ative abundance in organ}{Relative abundance {in}^{“} {Input}^{”}}$$

Validation of Barcoded Payload mRNA Stability

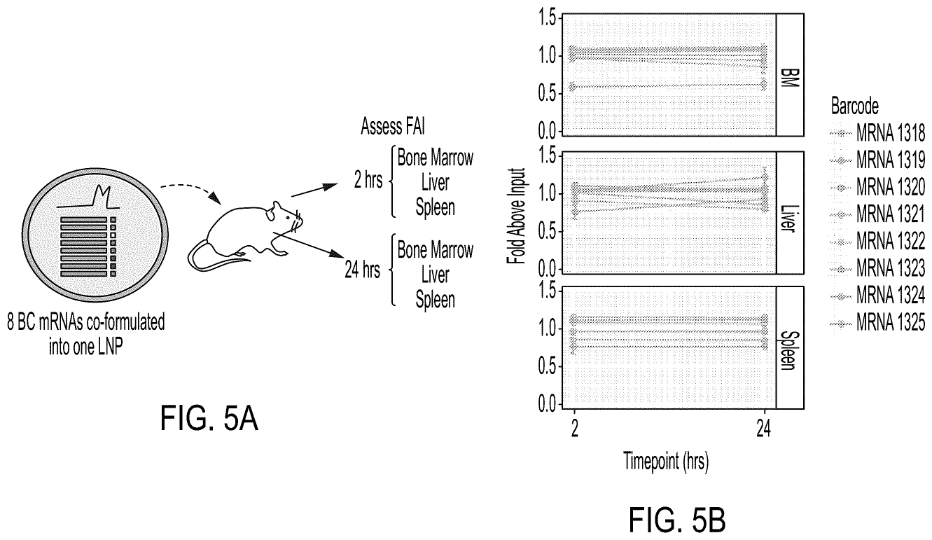

One LNP (Lipid A) encapsulated 8 barcoded payload mRNAs (mRNAs #1318-1325). The mRNA-loaded LNP was injected to mice. Liver, spleen and bone marrow were isolated from mice 2 hours and 24 hours after administration of the mRNA loaded LNP (FIG. 5A). The relative abundance of each barcoded payload mRNA was measured at 2 hours and 24 hours. The relative abundance of each payload barcoded mRNA was measured at 2 hours or 24 hours after injection to mice at a dose of 0.65 mg/kg. The FAI value for each barcode sequence in liver, spleen and bone marrow at 2 hours and 24 hours was determined. As shown in FIG. 5B, different 8nt barcoded mRNAs co-formulated into one LNP can be successfully sequenced and exhibited similar degradation data between 2 and 24 hours (all 8 barcodes within 15%). The results indicate that the presence of a barcode in the 3′UTR of the payload mRNA does not lead to differential degradation of the barcoded payload mRNA.

Correlation of the Input and Output of a Pooled Barcoded mRNA-LNPs

To test the correlation between the input and output abundance of barcodes in pooled LNPs encapsulating barcoded mRNAs, 8 LNPs (Lipid A) encapsulated the 8 different barcoded mRNA encoding base editor (mRNAs #1318-1325); each LNP encapsulated one barcoded mRNA. The 8 LNPs were pooled together at different concentrations (from 0.1% to 40%). The pool of 8 LNPs was injected into mice at 0.12 mg/kg and 0.8 mg/kg. The spleen and liver were isolated at 2 hours, 6 hours, and 24 hours post-administration. The FAI values were measured (data not shown). The barcode abundance indicated that there is a strong correlation between barcoded mRNA input and output abundance which is independent of injection dose, timepoint of the measures and tissue types being measured. The correlation validates the pooled LNPs formulated with different barcoded payload mRNAs for screening LNPs.

### LNP Dose and Timepoint Influences on FAI Readout

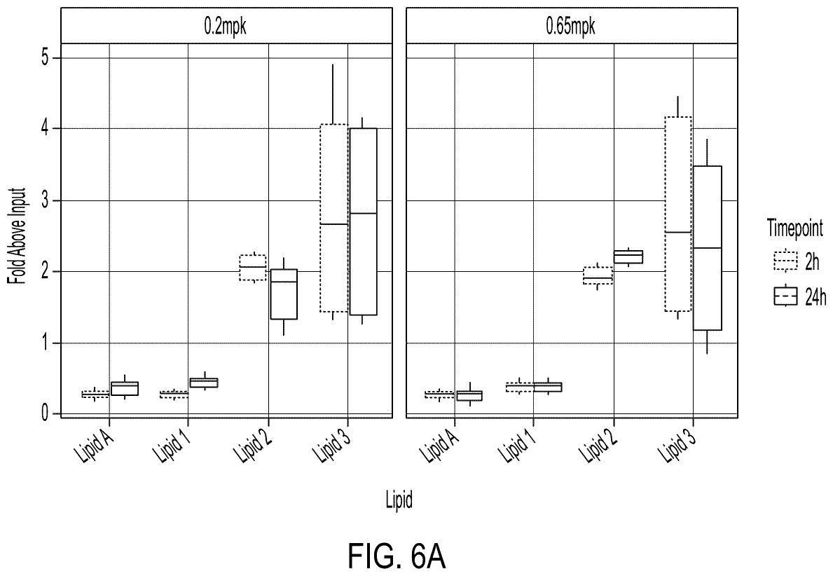

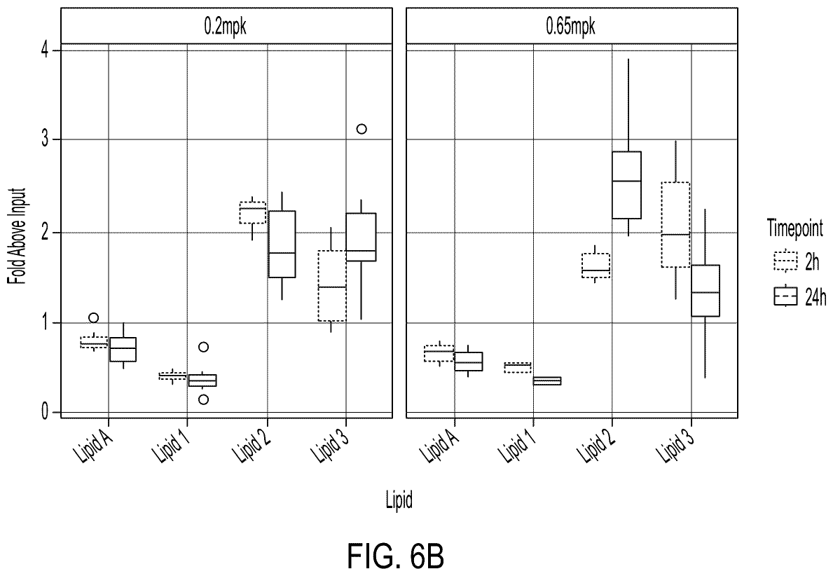

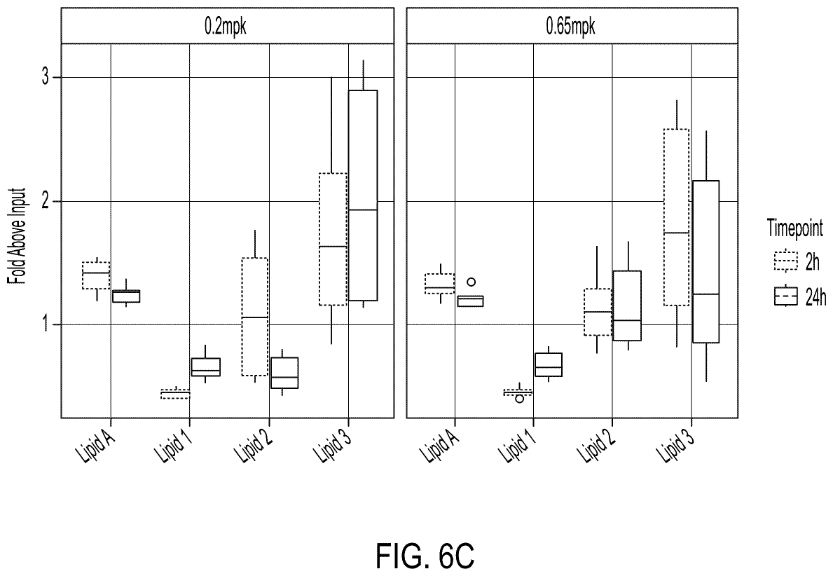

A pool of 8 LNPs representing 4 different formulations (each has two formulation replicates) was injected to mice at 0.2 mg/kg and 0.65 mg/kg total barcoded payload mRNAs. The spleen and bone marrow and liver were isolated at 2 hours or 24 hours after injection. The lipids used in the experiments are disclosed in the PCT patent application publications: WO 2017/075531 and WO 2021/141969, and the PCT application No. PCT/US2021/64339. The relative abundance was determined. The data show that neither timepoint nor dose impact LNP biodistribution in the spleen (FIG. 6A). In the bone marrow and liver (FIGS. 6B and 6C), it was also observed that neither time nor dose impact LNP biodistribution. Though there is some variance, these data suggest that LNP doses and timepoint do not adversely affect LNP biodistribution.

### Example 3: qPCR-Based Normalization Method of Barcoded Payload mRNA FAI

RT-qPCR data can normalize the FAI. By using RT-qPCR quantifications of both the barcode and a housekeeping gene, a comparison across tissues, time, dose, species and screens were performed. The FAI is multiplied by the linearized RT-qPCR data (2^{dCT}). The following formula is used to normalize the FAI using RT-qPCR data of the barcodes, thereby converting back to quantitative data (relative to a housekeeping gene).

$$log ( FAI ( x ) \cdot {2}^{dCTx} ) - log ( FAI ( y ) \cdot {2}^{dCTy} ) \qquad Formula II$$

As a general process, approximately the same amount of tissue (e.g., 10-20 mg) is extracted from RNAlater-preserved samples. The barcoded mRNAs and a housekeeping gene (ActB) in the sample are quantitated in a multiplex RT-qPCR. The relative amount of barcodes (“bc-mRNA”) to actB transcripts is represented by the difference in their cycle threshold (“Ct” or “CT”) values: 2^{(CtactB-Ctbc-mRNA)}. Assuming the housekeeping gene actB is expressed similarly in all tissues and cells being compared, Ct_{actB}-Ct_{bc-mPNA} can be thought of as representing the approximate number of barcodes per cell. Differences in actB signals are interpreted are differences in extraction yields. The same housekeeping gene across validation for different species (e.g., mouse, rat and non-human primate) is used to keep the results consistent. The flowchart in FIG. 7 shows RT-qPCR process to quantitate barcoded mRNAs.

Primers for gPCR

A pair of primers targeting the CDS region (forward) and downstream the barcode at the 3′UTR (reverse) and a probe sequence were designed for RT-qPCR. The primer positions was selected to avoid to detect endogenous RNA for alpha globin.

| | Forward primer |
|---|---|
| (SEQ ID NO. 62) | |
| | 5′AAGCGCAAAGTGTAGTTAAT3′ |
| | Reverse primer |
| (SEQ ID NO. 63) | |
| | 5′TTCCTACTCAGGCTTTATTCA3′ |
| | Probe sequence: |
| (SEQ ID NO. 64) | |
| | 5′TCTGGCCATGCCCTTCTTCT3′ |

The species-specific primers and probes for Beta Actin Housekeeping gene were also designed based the sequences of Mouse ActB: Mm02619580_g1; Rat ActB: Rn00667869_ml; and Cynomolgus macaque ActB: Mf04354341_g1. The same housekeeping gene was used across validation for all 3 species to keep data consistent.

The barcoded payload mRNA and Beta actin (ActB) primers and probes were tested in singleplex (only one primer set at a time) and multiplex (both primers in same reaction tube).

TABLE 8
RT-qPCR normalization

| Mouse | ActB efficiency | Barcoded payload mRNA efficiency |
|---|---|---|
| Singleplex | 103 | 112.7 |
| Multiplex | 102.4 | 107.7 |

Table 8 shows that the efficiencies between the payload mRNA and actB were consistent, within 90-110% for all reaction. There was no cross-reactivity occurred between Mouse, Rat, and Cynomologus macaques.

### RT-gPCR Based Normalization of FAI Data

The RT-qPCR based normalization of FAI data enables comparison between organs, time points, dose levels, independent screens and different species. The lipids used in the experiments are disclosed in the PCT patent application publications: WO 2017/075531 and WO 2021/141969, and the PCT application No. PCT/US2021/64339.

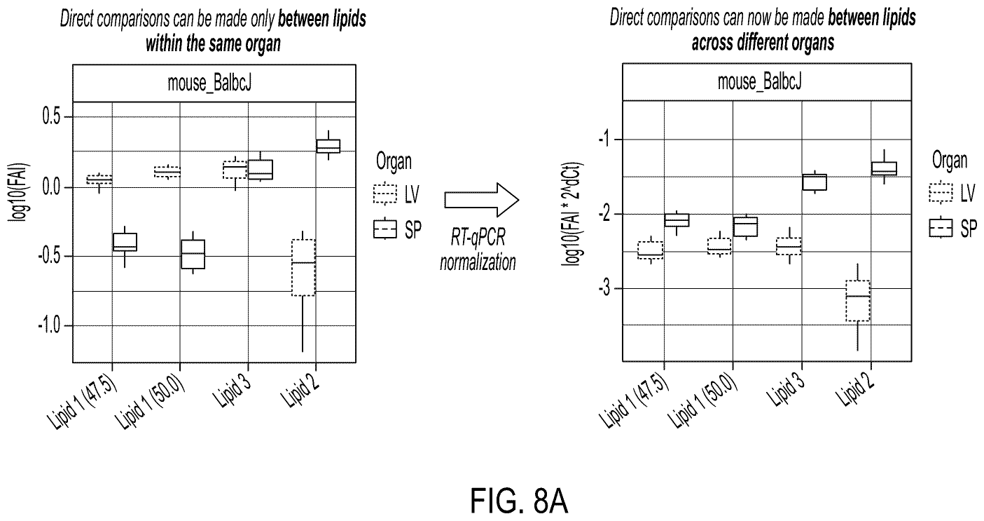

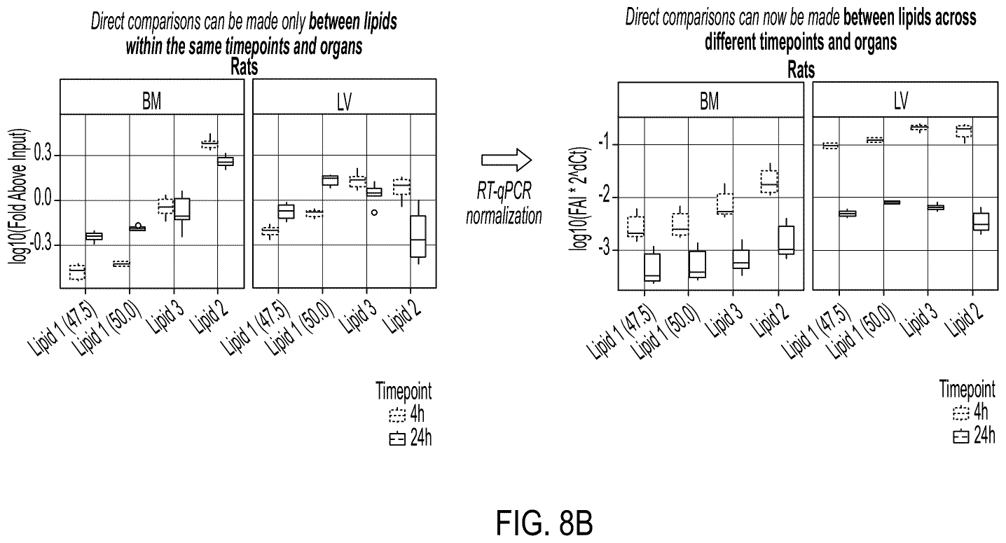

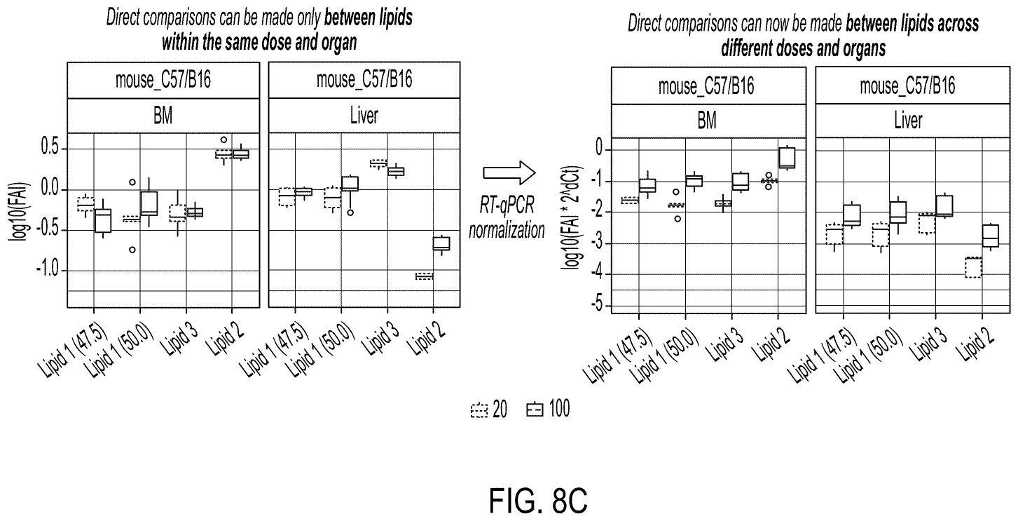

In one study, a pool containing 8 barcoded LNP formulations, representing 4 different LNP compositions (either different molar ratios or ionizable lipid structures) each formulated two times, was injected into Balb/c mice. At 24 hours post-administration, the mice were sacrificed at which time the liver and spleen were isolated. RNA was isolated from tissues and barcode FAI was determined by next-generation sequencing. Separately, the amount of barcoded mRNA (agnostic of barcode sequence) was determined via RT-qPCR normalizing barcoded mRNA to a housekeeping gene, Beta-Actin. The amount of barcoded mRNA in the tissue (relative to ActB in the same tissue) was used to normalize FAI to allow the comparison of LNP delivery to liver and spleen. FIG. 8A. indicates that by RT-qPCR normalization, the distributions of LNPs across different organs, (e.g., liver and spleen) can be directly compared.

In another study, a pool containing 8 barcoded LNP formulations, representing 4 different LNP compositions (either different molar ratios or ionizable lipid structures) each formulated two times, was injected into Sprague Dawley Rats. At 4 and 24 hours post-administration, the mice were sacrificed at which time the liver and bone marrow were isolated. RNA was isolated from tissues and barcode FAI was determined by next-generation sequencing. Separately, the amount of barcoded mRNA (agnostic of barcode sequence) was determined via RT-qPCR normalizing barcoded mRNA to a housekeeping gene, Beta-Actin. The amount of barcoded mRNA in the tissue (relative to ActB) was used to normalize FAI to allow the comparison of barcoded mRNA present in the liver and bone marrow at two time points. As shown in FIG. 8B, Comparisons between time points are not valid pre-normalization by RT-qPCR, while, after normalization, the LNP distribution at 24 hour is lower than 4 hours, which can identify clearance rate.

In another study, a pool containing 8 barcoded LNP formulations, representing 4 different LNP compositions (either different molar ratios or ionizable lipid structures) each formulated two times, was injected into C57BL6 mice at doses of 0.2 and 1.0 mg/kg. At 4 hours post-administration, the mice were sacrificed at which time the liver and bone marrow were isolated. RNA was isolated from tissues and barcode FAI was determined by next-generation sequencing. Separately, the amount of barcoded mRNA (agnostic of barcode sequence) was determined via RT-qPCR normalizing barcoded mRNA to a housekeeping gene, Beta-Actin. The amount of barcoded mRNA in the tissue (relative to ActB) was used to normalize FAI to allow the comparison of barcoded mRNA present in the liver and bone marrow in animals administered at two different doses. As shown in FIG. 8C, the RT-qPCR normalization can also allow dose comparison.

### Example 4: Barcoded Payload mRNA to Screen LNPs for Cell-Specificity of Tissue Level Readouts

A barcoded payload mRNA is designed to further add a miR-122 binding site at the 3′UTR; the miR-122 binding site is separate from the barcode at the 3′UTR of the payload mRNA, LNPs encapsulating the barcoded payload mRNAs comprising the miRNA-122 binding site are injected to mice. The liver is collected from the mice at 2 hours, 6 hours, 12 hours and 24 hours. The FAI values for each barcode in the liver are measured and normalized by RT-qPCR. Alternatively, hepatocytes from the liver are isolated and sorted, the FAI values for each barcode in hepatocytes are measured and normalized by RT-qPCR.

### EQUIVALENTS AND SCOPE

Those skilled in the art will recognize, or be able to ascertain using no more than routine experimentation, many equivalents to the specific embodiments of the invention described herein. The scope of the present invention is not intended to be limited to the above Description, but rather is as set forth in the following claims.

1. A method for screening nanoparticles for preferential delivery to a target tissue or cell, comprising:
(i) administering a plurality of nanoparticles having different lipid compositions to a non-human mammal, wherein individual nanoparticle encapsulates a payload mRNA comprising a 3′ untranslated region (3′UTR) and a barcode that correlates with the lipid composition of said individual nanoparticle;
(ii) measuring relative abundance of each barcode in one or more target tissues or cells of interest;
(iii) comparing the relative abundance of each barcode to its corresponding relative input abundance to determine fold above input (FAI) for each barcode; and
(iv) if the FAI of a barcode in a target tissue or cell is above a threshold, identifying the nanoparticle correlated with the barcode as a candidate nanoparticle suitable for preferential delivery to the target tissue or cell.

2. The method of claim 1, wherein the nanoparticle is a lipid nanoparticle.

3. The method of claim 1 or 2, wherein the payload mRNA does not comprise a unique molecular identifier (UMI).

4. The method of any one of claims 1-3, wherein the barcode is located in the 3′UTR region of the payload mRNA.

5. The method of any one of the preceding claims, wherein the barcode comprises 4-30, 6-25, or 6-20 nucleotides.

6. The method of claim 5, wherein the barcode comprises 8 or 20 nucleotides.

7. The method of any one of the preceding claims, wherein each barcode sequence has a hamming distance of 1, 2, 3, 4 or greater from any other barcode sequences.

8. The method of any one of the preceding claims, wherein the barcode sequence has minimal predicted AG.

9. The method of any one of the preceding claims, wherein the barcode minimally impacts mRNA stability.

10. The method of any one of the preceding claims, wherein the barcode sequence is contiguous, or non-contiguous.

11. The method of any one of the preceding claims, wherein the individual nanoparticle further encapsulates a second RNA molecule.

12. The method of claim 11, wherein the second RNA molecule is a guide RNA.

13. The method of claim 11 or 12, wherein the payload mRNA and the second RNA is at a mass ratio from 1:4 to 4:1, or at a mass ratio of 1:1, 1:2, 2:1, 1:3, 3:1, 1:4, or 4:1.

14. The method of any one of the preceding claims, wherein the payload mRNA comprises a microRNA binding site.

15. The method of claim 14, wherein the microRNA binding site leads to accelerated barcoded payload mRNA degradation in specific cell-types.

16. The method of any one of claims 14-15, wherein the microRNA-binding site is located at the 3′UTR but does not overlap with the barcode.

17. The method of any one of claims 14-16, wherein the microRNA-binding site and the barcode have minimal or zero sequence homology.

18. The method of any one of the preceding claims, wherein the payload mRNA encodes a nuclease, or variant thereof.

19. The method of claim 18, wherein the nuclease is a nucleobase editor, or variant thereof.

20. The method of claim 19, wherein the nucleobase editor is a cytidine base editor (CBE), an adenosine base editor (ABE), or variant thereof.

21. The method of claim 18, wherein the nuclease is a member of CRISPR-associated protein family, or variant thereof.

22. The method of claim 21, wherein the CRISPR-associated protein is Cas1, Cas2, Cas3, Cas4, Cas5, Cas6, Cas6, Cas7, Cas8, Cas9, Cas10, Cas11, Cas12, Cas13, Cse1, Cse2, Csf1, Csm2, Csn2, Csx10, Csx11, Csy1, Csy2, Csy3, C2c1, C2c2, C2c3, C2c4, C2c5, C2c8, C2c9, Cpf1, or Cmr5, or variant thereof.

23. The method of any one of the preceding claims, wherein the target tissues of interest are selected from the group consisting of liver, spleen, bone marrow, lung, brain, heart, kidney, eye, lymph, muscle, spine, stomach, intestine, pancreas, and combination thereof.

24. The method of any one of the preceding claims, wherein the target cells of interest are selected from the group consisting of endothelial cells, fibroblasts, epithelial cells, neurons, glia cells, immune cells, hepatocytes, lipocytes, muscular cells, differentiated cells, progenitor cells, stem cells, cancer cells, and combination thereof.

25. The method of any one of the preceding claims, wherein the abundance of payload mRNA is measuring by high throughput sequencing, next-generation sequencing, or deep sequencing.

26. The method of claim 25, wherein the abundance of each payload mRNA in target cells of interest is determined by single-cell RNA sequencing (scRNA-seq), or by assessing transcription profiles.

27. The method of any one of the preceding claims, wherein the abundance of each payload mRNA is measuring by RT-qPCR.

28. The method of any one of the preceding claims, wherein the relative abundance of each barcode in one or more tissues or cells is measured at 2 hours, 4 hours, 6 hours, 8 hours, 10 hours, 12 hours, 24 hours and/or 72 hours after administration.

29. The method of any one of the preceding claims, wherein the FAI is determined by the following formula:
$$FAI = \frac{R e l ative abundance in organ}{Relative abundance {in}^{“} {Input}^{”}}$$

30. The method of any one of claims 27-29, wherein the FAI is normalized according to the following formula:
$$log ( FAI ( x ) \cdot {2}^{dCTx} ) - log ( FAI ( y ) \cdot {2}^{dCTy} )$$
wherein FAI(x) is the FAI of a barcoded payload mRNA in Sample x; FAI(y) is the FAI(y) is the FAI of the barcoded payload mRNA in Sample y; dCTx is the difference of the cycle threshold (CT) values of a housekeeping gene and the barcoded payload mRNA in Sample x;
and dCTy is the difference of the cycle threshold (CT) values of the housekeeping gene and the barcoded payload mRNA in Sample y.

31. The method of any one of the preceding claims, wherein the threshold is pre-determined.

32. The method of claim 31, wherein the threshold is 2, 3, 4, 5, 6, 8, 10, 12, 14, 16, 18, 20, 25, 30, 35, 40, 50, 60, 70, 80, 90, 100, 200, 300, 400, 500, 600, 700, 800, 900, or 1000.

33. The method of anyone of the preceding claims, wherein the threshold is determined based on a control run in parallel.

34. The method of any one of the preceding claims, wherein the non-human mammal is a mouse, a rat, a rabbit, a pig, a goat, or a non-human primate.

35. The method of any one of the preceding claims, wherein the method further comprising validating the candidate LNP in a second non-human mammal, or in a biological system, wherein the second non-human mammal is a mouse, a rat, a rabbit, a pig, a goat, or a non-human primate.

36. The method of claim 35, wherein the second non-human mammal is a different species from the screening.

37. The method of claim 35, wherein the biological system is a cell-based system, or a disease model.

38. The method of any one of claims 2-37, wherein the LNP comprises at least one ionizable lipid, at least one helper lipid, at least one cholesterol-based and/or at least one PEGylated-lipid.

39. A method for screening lipid nanoparticles for preferential delivery to a target tissue or cell, comprising:
(i) administering a plurality of nanoparticles having different lipid compositions to a non-human mammal, wherein individual nanoparticle encapsulates a payload mRNA comprising a barcode that correlates with the lipid composition of said individual nanoparticle, and wherein the payload mRNA does not comprise a unique molecular identifier (UMI); and
(ii) determining relative abundance of each barcode in one or more target tissues or cells of interest as compared to a reference, thereby identifying a candidate nanoparticle suitable for preferential delivery to a target tissue or cell.

40. A nanoparticle for preferential delivery to a target tissue or cell identified using a method of any one of the preceding claims.

41. An mRNA comprising a barcode at the 3′ untranslated region (3′ UTR), wherein the mRNA does not comprise a unique molecular identifier (UMI).

42. An mRNA comprising a barcode at the 3′ untranslated region (3′ UTR) and a microRNA binding site.

43. The mRNA of any one of claim 41 or 42, wherein the barcode comprises 4-30, 6-25, or 6-20 nucleotides.

44. The mRNA of claim 43, wherein the barcode comprises 8 or 20 nucleotides.

45. The mRNA of any one of claims 41-44, wherein the barcode sequence has minimal predicted AG.

46. The mRNA of any one of claims 41-45, wherein the barcode minimally impacts mRNA stability.

47. The mRNA of any one of claims 41-46, wherein the barcode sequence is contiguous, or non-contiguous.

48. The mRNA of any one of claims 42-47, wherein the barcode and the microRNA binding site does not overlap.

49. The mRNA of any one of claims 42-48, wherein the barcode and the microRNA binding site have minimal or zero sequence homology.

50. The mRNA of any one of claims 41-49, wherein the mRNA encodes a nuclease, or variant thereof.

51. The mRNA of claim 50, wherein the nuclease is a nucleobase editor, or variant thereof.

52. The mRNA of claim 51, wherein the nucleobase editor is a cytidine base editor (CBE), an adenosine base editor (ABE), or variant thereof.

53. The mRNA of claim 50, wherein the nuclease is a member of CRISPR-associated protein family, or variant thereof.

54. The mRNA of claim 53, wherein the CRISPR-associated protein is Cas1, Cas2, Cas3, Cas4, Cas5, Cas6, Cas6, Cas7, Cas8, Cas9, Cas10, Cas11, Cas12, Cas13, Cse1, Cse2, Csf1, Csm2, Csn2, Csx10, Csx11, Csy1, Csy2, Csy3, C2c1, C2c2, C2c3, C2c4, C2c5, C2c8, C2c9, Cpf1, or Cnr5, or variant thereof.

55. A nanoparticle encapsulating an mRNA of any one of claims 41-55.

56. A method for producing a barcoded mRNA comprising,
i) amplifying a linear protein coding nucleic acid sequence having a 3′UTR using a 5′ primer and a 3′ primer having a sequence specific to the 3′UTR, wherein the 3′ primer further comprises a nucleic acid barcode sequence; and
ii) transcribing in vitro the amplified linear protein coding nucleic acid sequence.

57. The method of claim 56, wherein the linear protein coding nucleic acid sequence comprises, in order from the 5′ to 3′ end, a T7 promoter, a 5′UTR, an open reading frame, and the 3′UTR, and wherein the linear protein coding nucleic acid sequence does not comprise a nucleic acid barcode sequence.

58. The method of claim 57, wherein the 5′ primer is specific to the T7 promoter and wherein the 3′ primer comprises, in order from the 5′ to 3′ end, the sequence specific to the 3′UTR, the nucleic acid barcode, and a polyA tail.

59. The method of claims 56-58, wherein the linear protein coding nucleic acid sequence is produced from a plasmid.

60. The method of claims 56-59, wherein the 3′ primer comprises the sequence of any one of SEQ ID NOs. 54-61.
